(19) United States
(12) Patent Application Publication
(10) Pub. No.: US 2026/0184787 A1
(43) Pub. Date: Jul. 2, 2026

(54) COMBINATION THERAPY WITH ANTI-CD19/ANTI-CD28 BISPECIFIC ANTIBODY

(71) Applicant: Hoffmann-La Roche Inc., Little Falls, NJ (US)

(72) Inventors: Sylvia HERTER, Baden-Rütihof (CH); Thomas HOFER, Zürich (CH); Christian KLEIN, Bonstetten (CH); Johannes SAM, Baden (CH); Pablo UMAÑA, Lachen (CH)

(73) Assignee: Hoffmann-La Roche Inc., Little Falls, NJ (US)

(21) Appl. No.: 19/126,612

(22) PCT Filed: Nov. 1, 2023
(86) PCT No.: PCT/EP2023/080438

§ 371 Date: May 1, 2025

(30) Foreign Application Priority Data
Nov. 3, 2022 (EP) 22205429.8

(51) Int. Cl. C07K 16/28; A61K 38/00; A61K 39/00; A61P 35/00; C07K 14/705

(52) U.S. Cl. CPC C07K 16/2809; A61P 35/00; C07K 14/70575; C07K 16/2803; C07K 16/2818; C07K 16/2887; A61K 38/00; A61K 2039/507; A61K 2039/545; C07K 2317/31; C07K 2317/52; C07K 2317/522; C07K 2317/55; C07K 2317/565; C07K 2317/75; C07K 2319/00

(57) ABSTRACT

The present invention relates to combination therapies employing an anti-CD20/anti-CD3 bispecific antibody in combination with an anti-CD19/anti-CD28 bispecific antibody and a CD19-targeted 4-1BB (CD137) agonist and the use of these combination therapies for the treatment of B-cell cancer such as diffuse large B cell lymphoma (DLBCL).

### FIELD OF THE INVENTION

The present invention relates to combination therapies employing anti-CD20/anti-CD3 bispecific antibody in combination with an anti-CD19/anti-CD28 bispecific antibody and a CD19-targeted 4-1BB (CD137) agonist and the use of these combination therapies for the treatment of a B cell proliferative disorder such as diffuse large B cell lymphoma (DLBCL).

### BACKGROUND

B-cell proliferative disorders describe a heterogeneous group of malignancies that includes both leukemias and lymphomas. Lymphomas develop from lymphatic cells and include two main categories: Hodgkin lymphomas (HL) and the non-Hodgkin lymphomas (NHL). In the United States, lymphomas of B cell origin constitute approximately 80-85% of all non-Hodgkin lymphoma cases, and there is considerable heterogeneity within the B-cell subset, based upon genotypic and phenotypic expression patterns in the B-cell of origin. For example, B cell lymphoma subsets include the slow-growing indolent and incurable diseases, such as Follicular lymphoma (FL) or chronic lymphocytic leukemia (CLL), as well as the more aggressive subtypes, mantle cell lymphoma (MCL) and diffuse large B cell lymphoma (DLBCL). Diffuse large B-cell lymphoma (DLBCL) is the most common type of NHL accounting for approximately 30%-40% of all NHL diagnosis, followed by follicular lymphoma (FL; 20%-25% of all NHL diagnosis) and mantle cell lymphoma (MCL; 6%-10% of all NHL diagnosis). B-cell chronic lymphocytic leukemia (CLL) is the most common leukemia in adults, with approximately 15,000 new cases per year in the United States (American Cancer Society 2015). Despite the availability of various agents for the treatment of B-cell proliferative disorders, there is an ongoing need for development of safe and effective therapies to prolong remission and improve cure rates in patients.

An anti-CD20/anti-CD3 bispecific antibody is a molecule that targets CD20 expressed on B cells and CD3 epsilon chain (CD3ε) present on T cells. Simultaneous binding leads to T-cell activation and T-cell mediated killing of B cells. In the presence of CD20 expressing B cells, whether circulating or tissue resident, pharmacologically active doses of anti-CD20/anti-CD3 bispecific antibody will trigger T-cell activation and associated cytokine release. Glofitamab is a T cell bispecific (TCB) antibody targeting CD20 expressed on B cells and CD3 epsilon chain (CD3ε) present on T cells. Parallel to B cell depletion in the peripheral blood, anti-CD20/anti-CD3 bispecific antibody leads to a transient decrease of T cells in the peripheral blood within 24 hours after the first administration and to a peak in cytokine release, followed by rapid T-cell recovery and return of cytokine levels to baseline within 72 hours. Thus, in order to achieve complete elimination of tumor cells, there is a need of additional agents that conserve T-cell activation and deliver durable immune response to cancer cells.

4-1BB (CD137) is an inducible member of the tissue necrosis factor (TNF) receptor superfamily expressed by activated T cells. Many other immune cells also express 4-1BB, including NK cells, B cells, NKT cells, monocytes, neutrophils, mast cells, dendritic cells (DCs) and cells of non-hematopoietic origin such as endothelial and smooth muscle cells. Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines. 4-1BB ligand (4-1BBL or CD137L) was identified in 1993. It has been shown that expression of 4-1BBL was restricted on professional antigen presenting cells (APC) such as B-cells, DCs and macrophages. Inducible expression of 4-1BBL is characteristic for T-cells, including both αβ and γδ T-cell subsets, and endothelial cells.

Co-stimulation through the 4-1BB receptor (for example by 4-1BBL ligation) activates multiple signaling cascades within the T cell (both CD4⁺ and CD8⁺ subsets), powerfully augmenting T cell activation. In combination with TCR triggering, agonistic 4-1BB-specific antibodies enhance proliferation of T-cells, stimulate lymphokine secretion and decrease sensitivity of T-lymphocytes to activation-induced cells death. This mechanism was further advanced as the first proof of concept in cancer immunotherapy. In a preclinical model administration of an agonistic antibody against 4-1BB in tumor bearing mice led to potent anti-tumor effect. Later, accumulating evidence indicated that 4-1BB usually exhibits its potency as an anti-tumor agent only when administered in combination with other immunomodulatory compounds, chemotherapeutic reagents, tumor-specific vaccination or radiotherapy (Bartkowiak and Curran, 2015).

Signaling of the TNFR-superfamily needs cross-linking of the trimerized ligands to engage with the receptors, so does the 4-1BB agonistic antibodies which require wild type Fc-binding. However, systemic administration of 4-1BB-specific agonistic antibodies with the functionally active Fc domain resulted in influx of CD8⁺ T-cells associated with liver toxicity that is diminished or significantly ameliorated in the absence of functional Fc-receptors in mice. In the clinic, an Fc-competent 4-1BB agonistic Ab (BMS-663513) (NCT00612664) caused a grade 4 hepatitis leading to termination of the trial. Therefore, there is a need for effective and safer 4-1BB agonists. An example thereof is an antigen binding molecule composed of a trimeric and thus biologically active 4-1BB ligand and an antigen binding domain specific for the tumor antigen CD19 and a silent Fc domain (herein named as CD19-4-1BBL). This construct has been described in WO 2016/075278 and replaces unspecific FcγR-mediated crosslinking responsible for Fc-mediated toxicity, by CD19-targeted B cell specific crosslinking.

CD19 is an ideal target for immunotherapy of B-cell malignancies as it is expressed on the surface of B-cells and is almost specific to these cells. CD19 is more broadly expressed than CD20 on B cells during the B cell development, so typically a CD20 positive cell will also expressed CD19. During differentiation of B cells towards plasma cells (antibody-secreting cells), B cells down-regulates CD20 expression. Sometimes, B cell lymphomas also down-regulate CD20 expression, but remain positive for CD19. Therefore, targeting both CD19 and CD20 would cover broadly the diseased B cells in lymphomas, which might also deviate the selection pressure from CD20 to both CD19 and CD20. Though it is not known if CD19 contributes directly to B cell carcinogenesis, its expression is highly conserved on most B cell tumors such as acute lymphoblastic leukemias (ALL), chronic lymphocytic leukemias (CLL) and B cell lymphomas. In acute leukemias CD19 is steadily and continuously expressed on almost all subtypes while only a small number of leukemias express CD20.

CD28 is the founding member of a subfamily of costimulatory molecules characterized by paired V-set immunoglobulin superfamily (IgSF) domains attached to single transmembrane domains and cytoplasmic domains that contain critical signaling motifs. Other members of the subfamily include ICOS, CTLA-4, PD1, PD1H, TIGIT, and BTLA. CD28 expression is restricted to T cells and prevalent on all naïve and a majority of antigen-experienced subsets, including those that express PD-1 or CTLA-4. CD28 and CTLA-4 are highly homologous and compete for binding to the same B7 molecules CD80 and CD86, which are expressed on dendritic cells, B cells, macrophages, and tumor cells. The higher affinity of CTLA-4 for the B7 family of ligands allows CTLA-4 to outcompete CD28 for ligand binding and suppress effector T cells responses. In contrast, PD-1 was shown to inhibit CD28 signaling by in part dephosphorylating the cytoplasmic domain of CD28. Recent evidence demonstrates that the anti-cancer effects of PD-L1/PD-1 and CTLA-4 checkpoint inhibitors depend on CD28. CD28 is constitutively expressed on the cell surface of CD4- and CD8-positive T cells. Upon provision of a so-called signal 1 via TCR or CD3 engagement, co-stimulation via CD28 activates multiple signaling cascades within the T cell to enhance the T cell-mediated immune response. CD28-mediated signal 2 is thought to occur via co-clustering at the immune synapse. CD28 agonistic antibodies can be administered alongside a signal 1 provider such as an antibody targeting CD3 to enhance the immune response. Immune stimulation is a complex cascade, and an out of control response has significant dangers. A phase I study of the human CD28 antibody TGN1412 resulted in a life-threatening cytokine storm in 2006. In contrast to TGN1412, anti-CD19/anti-CD28 bispecific antibodies avoid autonomous T cell activation because only in the presence of CD19-expressing tumor cells and a signal 1 via TCR or CD3 engagement T cell proliferation, cytokine secretion and tumor cell killing is induced.

With the recent development of second and later generations of T cells genetically engineered to express chimeric antigen receptors (CAR-T) for patients with CD19-positive malignancies, CD28 has regained major interest as an immunotherapeutic target. The proof of concept has been shown in several trials showing response rates from 64% to 82% in heavily pre-treated NHL patients using autologous anti-CD19-directed CAR-T therapy including CD28 signaling domains. However, there are major limitations to CAR-T cell therapy that still must be addressed including life-threatening CAR-T cell-associated toxicities inhibition and resistance in B cell malignancies, adverse events post-infusion such as cytokine release syndrome (CRS) and neurotoxicity, and host rejection of non-human CARs. General barriers include cellular manufacturing limitations, baseline quality of the T cells and time to infusion. For an autologous CAR-T cell therapy it will take 4 to 6 weeks until the engineered CAR T cells are prepared from the patient's T cells and the delay may compromise the outcome of the therapy. In contrast, bispecific antibodies are available off-the-shelf.

Given that current standard-of-care treatments are not able to cure all patients suffering from B-cell proliferative disorders, there is a clear need to develop potent and specific new therapies.

### SUMMARY OF THE INVENTION

The present invention relates to anti-CD20/anti-CD3 bispecific antibodies and their use in combination with an anti-CD19/anti-CD28 bispecific antibody and a CD19-targeted 4-1BB (CD137) agonist for use in a combination therapy for the treatment of cancer, in particular for the treatment of B-cell proliferative disorders. It has been found that the combination therapy described herein is more effective in inhibiting tumor growth and eliminating tumor cells than treatment with the anti-CD20/anti-CD3 bispecific antibodies in combination with an anti-CD19/anti-CD28 bispecific antibody alone or in combination with a CD19-targeted 4-1BB (CD137) agonist alone.

In one aspect, the invention provides an anti-CD20/anti-CD3 bispecific antibody in combination with an anti-CD19/anti-CD28 bispecific antibody and a CD19-targeted 4-1BB (CD137) agonist for use in a combination therapy for the treatment of B-cell proliferative disorders.

In another aspect, the invention provides for the use of an anti-CD20/anti-CD3 bispecific antibody in combination with an anti-CD19/anti-CD28 bispecific antibody and an anti-CD19-targeted 4-1BB (CD137) agonist in the manufacture of a medicament for use in a combination therapy for the treatment of B-cell proliferative disorders.

In yet another aspect, the invention provides a method of treating B-cell cancer in an individual in need thereof comprising administering to said individual a combination therapy comprising an anti-CD20/anti-CD3 bispecific antibody in combination with an anti-CD19/anti-CD28 bispecific antibody and an anti-CD19-targeted 4-1BB (CD137) agonist.

In a further aspect, the invention provides a kit for use in a combination therapy comprising a first medicament comprising an anti-CD20/anti-CD3 bispecific antibody, a second medicament comprising an anti-CD19/anti-CD28 bispecific antibody and a third medicament comprising a CD19-targeted 4-1BB (CD137) agonist, and optionally further comprising a package insert comprising instruction for administration of the first medicament in combination with the second medicament for treating cancer in an individual.

In another aspect, provided is a medicament comprising an anti-CD20/anti-CD3 bispecific antibody for treating B-cell proliferative disorders, wherein the anti-CD20/anti-CD3 bispecific antibody is used in combination with an anti-CD19/anti-CD28 bispecific antibody and a CD19-targeted 4-1BB (CD137) agonist.

In a particular aspect, provided is the anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein before, wherein the combination therapy comprises a first treatment regimen with the anti-CD20/anti-CD3 bispecific antibody in combination with an anti-CD19/anti-CD28 bispecific antibody and a second treatment regimen anti-CD20/anti-CD3 bispecific antibody in combination with CD19-targeted 4-1BB (CD137) agonist.

In one aspect, the first treatment regimen comprises 1 to 5 treatment cycles and the second treatment regimen starts with the following treatment cycle. In another aspect, the first treatment regimen comprises 1 to 5 treatment cycles and the second treatment regimen starts with the following treatment cycle. In one aspect, the first treatment regimen comprises 4 treatment cycles and the second treatment regimen starts with the treatment cycle 5. In one aspect, there is a time interval of one week between the end of the first treatment regimen and the start of the second treatment regimen.

In all of these aspects, provided is the anti-CD20/anti-CD3 bispecific antibody in combination with an anti-CD19/anti-CD28 bispecific antibody and a CD19-targeted 4-1BB (CD137) agonist for use, the use, the method, the kit or the medicament, wherein a pretreatment with an Type II anti-CD20 antibody, preferably obinutuzumab, is performed prior to the combination therapy, and wherein the period of time between the pretreatment and the combination therapy is sufficient for the reduction of B-cells in the individual in response to the Type II anti-CD20 antibody, preferably obinutuzumab.

In one aspect of the invention, the CD19-targeted 4-1BB agonist comprises three ectodomains of 4-1BBL each comprising an amino acid sequence selected from the group consisting of SEQ ID NO:1, SEQ ID NO: 2, SEQ ID NO:3, SEQ ID NO:4, SEQ ID NO:5, SEQ ID NO: 6, SEQ ID NO:7, SEQ ID NO:8 and SEQ ID NO:9. More particularly, the ectodomains of 4-1BBL comprise an amino acid sequence of SEQ ID NO:5. In one aspect, the CD19-targeted 4-1BB agonist comprises a Fc domain, particularly an IgG1 or IgG4 Fc domain, that comprises one or more amino acid substitution that reduces or eliminates binding to an Fc receptor and/or effector function. More particularly, the CD19-targeted 4-1BB agonist comprises an IgG1 Fc domain comprising the amino acid substitutions L234A, L235A and P329G (EU numbering according to Kabat).

In a further aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein before, wherein the CD19-targeted 4-1BB agonist comprises an antigen binding domain capable of specific binding to CD19 comprising a heavy chain variable region (V_{H}CD19) comprising (i) CDR-H1 comprising the amino acid sequence of SEQ ID NO: 10, (ii) CDR-H2 comprising the amino acid sequence of SEQ ID NO:11, and (iii) CDR-H3 comprising the amino acid sequence of SEQ ID NO: 12, and a light chain variable region (V_{L}CD19) comprising (iv) CDR-L1 comprising the amino acid sequence of SEQ ID NO:13, (v) CDR-L2 comprising the amino acid sequence of SEQ ID NO:14, and (vi) CDR-L3 comprising the amino acid sequence of SEQ ID NO: 15. In particular, the CD19-targeted 4-1BB agonist comprises an antigen binding domain capable of specific binding to CD19 comprising a heavy chain variable region (V_{H}CD19) comprising an amino acid sequence of SEQ ID NO: 16 and a light chain variable region (V_{L}CD19) comprising an amino acid sequence of SEQ ID NO:17.

In another aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding paragraphs, wherein the CD19-targeted 4-1BB agonist comprises
(a) a first polypeptide, comprising (a1) the first ectodomain of 4-1BBL or fragment thereof, fused at its C-terminus to the N-terminus of the second ectodomain of 4-1BBL or fragment thereof, (a2) the second ectodomain of 4-1BBL or fragment thereof, fused at its C-terminus to the N-terminus of the CL domain, (a3) the CL domain, fused at its C-terminus to the N-terminus of one of the subunits (e.g. the first subunit) of the Fc domain, and (a4) one of the subunits (e.g. the first subunit) of the Fc domain;
(b) a second polypeptide, comprising (b1) the third ectodomain of 4-1BBL or fragment thereof, fused at its C-terminus to the N-terminus of the CH1 domain, and (b2) the CH1 domain;
(c) a third polypeptide, comprising (c1) the heavy chain of the Fab molecule that binds to CD19, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising the light chain of the Fab molecule that binds to CD19.

In one aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein before, wherein the CD19-targeted 4-1BB agonist comprises a first polypeptide comprising an amino acid sequence that is at least about 95%, 96%, 97%, 98%, 99% or 100% identical to the amino acid sequence of SEQ ID NO: 18, a second polypeptide comprising an amino acid sequence that is at least about 95%, 96%, 97%, 98%, 99% or 100% identical to the amino acid sequence of SEQ ID NO: 19, a third polypeptide comprising an amino acid sequence that is at least about 95%, 96%, 97%, 98%, 99% or 100% identical to the amino acid sequence of SEQ ID NO: 20, and a fourth polypeptide comprising an amino acid sequence that is at least about 95%, 96%, 97%, 98%, 99% or 100% identical to the amino acid sequence of SEQ ID NO: 21. More particularly, the CD19-targeted 4-1BB agonist comprises a first polypeptide comprising an amino acid sequence of SEQ ID NO: 18, a second polypeptide comprising an amino acid sequence of SEQ ID NO: 19, a third polypeptide comprising an amino acid sequence of SEQ ID NO: 20, and a fourth polypeptide comprising an amino acid sequence of SEQ ID NO: 21.

In a further aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as disclosed herein before, wherein the anti-CD20/anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}CD20) and a light chain variable region (V_{L}CD20). In one aspect, the first antigen binding domain comprises a heavy chain variable region (V_{H}CD3) comprising CDR-H1 sequence of SEQ ID NO:22, CDR-H2 sequence of SEQ ID NO:23, and CDR-H3 sequence of SEQ ID NO:24; and/or a light chain variable region (V_{L}CD3) comprising CDR-L1 sequence of SEQ ID NO:25, CDR-L2 sequence of SEQ ID NO:26, and CDR-L3 sequence of SEQ ID NO:27. In one aspect, the first antigen binding domain comprises a heavy chain variable region (V_{H}CD3) comprising the amino acid sequence of SEQ ID NO:28 and/or a light chain variable region (V_{L}CD3) comprising the amino acid sequence of SEQ ID NO:29. In one aspect, the second antigen binding domain comprises a heavy chain variable region (V_{H}CD20) comprising CDR-H1 sequence of SEQ ID NO:30, CDR-H2 sequence of SEQ ID NO:31, and CDR-H3 sequence of SEQ ID NO:32, and/or a light chain variable region (V_{L}CD20) comprising CDR-L1 sequence of SEQ ID NO:33, CDR-L2 sequence of SEQ ID NO:34, and CDR-L3 sequence of SEQ ID NO:35. In one aspect, the second antigen binding domain comprises a heavy chain variable region (V_{H}CD20) comprising the amino acid sequence of SEQ ID NO:36 and/or a light chain variable region (V_{L}CD20) comprising the amino acid sequence of SEQ ID NO:37.

In a further aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as disclosed herein before, wherein the anti-CD20/anti-CD3 bispecific antibody comprises a third antigen binding domain that binds to CD20. In one particular aspect, the anti-CD20/anti-CD3 bispecific antibody comprises a first polypeptide comprising an amino acid sequence that is at least about 95%, 96%, 97%, 98%, 99% or 100% identical to the amino acid sequence of SEQ ID NO: 38, a second polypeptide comprising an amino acid sequence that is at least about 95%, 96%, 97%, 98%, 99% or 100% identical to the amino acid sequence of SEQ ID NO: 39, a third polypeptide comprising an amino acid sequence that is at least about 95%, 96%, 97%, 98%, 99% or 100% identical to the amino acid sequence of SEQ ID NO: 40, and a fourth and fifth polypeptide both comprising an amino acid sequence that is at least about 95%, 96%, 97%, 98%, 99% or 100% identical to the amino acid sequence of SEQ ID NO: 41. More particularly, the anti-CD20/anti-CD3 bispecific antibody comprises a first polypeptide comprising an amino acid sequence of SEQ ID NO: 38, a second polypeptide comprising an amino acid sequence of SEQ ID NO: 39, a third polypeptide comprising an amino acid sequence of SEQ ID NO: 40, and a fourth and fifth polypeptide both comprising an amino acid sequence of SEQ ID NO: 41. More particularly, the anti-CD20/anti-CD3 bispecific antibody is glofitamab.

In one aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as disclosed herein before, wherein the anti-CD19/anti-CD28 bispecific antibody comprises a first antigen binding domain comprising a heavy chain variable region (V_{H}CD28) and a light chain variable region (V_{L}CD28), and a second antigen binding domain comprising a heavy chain variable region (V_{H}CD19) and a light chain variable region (V_{L}CD19). In one aspect, the anti-CD19/anti-CD28 bispecific antibody comprises a first antigen binding domain comprising a heavy chain variable region (V_{H}CD28) comprising CDR-H1 sequence of SEQ ID NO:42, CDR-H2 sequence of SEQ ID NO:43, and CDR-H3 sequence of SEQ ID NO:44, and/or a light chain variable region (V_{L}CD20) comprising CDR-L1 sequence of SEQ ID NO:45, CDR-L2 sequence of SEQ ID NO:46, and CDR-L3 sequence of SEQ ID NO:47. In one aspect, the anti-CD19/anti-CD28 bispecific antibody comprises a first antigen binding domain comprising a heavy chain variable region (V_{H}CD28) comprising the amino acid sequence of SEQ ID NO:48 and/or a light chain variable region (V_{L}CD28) comprising the amino acid sequence of SEQ ID NO:49. In one aspect, the anti-CD19/anti-CD28 bispecific antibody comprises a second antigen binding domain comprising a heavy chain variable region (V_{H}CD19) comprising CDR-H1 sequence of SEQ ID NO: 10, CDR-H2 sequence of SEQ ID NO:11, and CDR-H3 sequence of SEQ ID NO: 12; and/or a light chain variable region (V_{L}CD19) comprising CDR-L1 sequence of SEQ ID NO:13, CDR-L2 sequence of SEQ ID NO:14, and CDR-L3 sequence of SEQ ID NO: 15. In one aspect, the anti-CD19/anti-CD28 bispecific antibody comprises a second antigen binding domain comprising a heavy chain variable region (V_{H}CD19) comprising the amino acid sequence of SEQ ID NO: 16 and/or a light chain variable region (V_{L}CD19) comprising the amino acid sequence of SEQ ID NO: 17.

In one aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein before, wherein the anti-CD19/anti-CD28 bispecific antibody comprises a Fc domain, particularly an IgG1 or IgG4 Fc domain, that comprises one or more amino acid substitution that reduces or eliminates binding to an Fc receptor and/or effector function. More particularly, the anti-CD19/anti-CD28 bispecific antibody comprises an IgG1 Fc domain comprising the amino acid substitutions L234A, L235A and P329G (EU numbering according to Kabat).

In one particular aspect, the anti-CD19/anti-CD28 bispecific antibody comprises a first polypeptide comprising an amino acid sequence that is at least about 95%, 96%, 97%, 98%, 99% or 100% identical to the amino acid sequence of SEQ ID NO: 50, a second polypeptide comprising an amino acid sequence that is at least about 95%, 96%, 97%, 98%, 99% or 100% identical to the amino acid sequence of SEQ ID NO: 51, a third polypeptide comprising an amino acid sequence that is at least about 95%, 96%, 97%, 98%, 99% or 100% identical to the amino acid sequence of SEQ ID NO: 52, and a fourth polypeptide both comprising an amino acid sequence that is at least about 95%, 96%, 97%, 98%, 99% or 100% identical to the amino acid sequence of SEQ ID NO: 53. More particularly, the anti-CD19/anti-CD28 bispecific antibody comprises a first polypeptide comprising an amino acid sequence of SEQ ID NO: 50, a second polypeptide comprising an amino acid sequence of SEQ ID NO: 51, a third polypeptide comprising an amino acid sequence of SEQ ID NO: 52, and a fourth polypeptide comprising an amino acid sequence of SEQ ID NO: 53.

In a further aspect, the invention provides the anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein before, wherein the combination therapy is administered at intervals from about one week to three weeks.

In one aspect, provided is an anti-CD20/anti-CD3 bispecific antibody in combination with an anti-CD19/anti-CD28 bispecific antibody and a CD19-targeted 4-1BB (CD137) agonist for use, the use, the method, the kit or the medicament as disclosed herein before, wherein the B-cell proliferative disorder is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), Multiple myeloma (MM) and Hodgkin lymphoma (HL). In one particular aspect, the B-cell proliferative disorder is diffuse large B-cell lymphoma (DLBCL).

In a further aspect, the invention provides an anti-CD20/anti-CD3 bispecific antibody in combination with an anti-CD19/anti-CD28 bispecific antibody and a CD19-targeted 4-1BB (CD137) agonist for use, the use, the method, the kit or the medicament as disclosed herein before, wherein the anti-CD20/anti-CD3 bispecific antibody, the anti-CD19/anti-CD28 bispecific antibody and the CD19-targeted 4-1BB (CD137) agonist are administered intravenously. In another aspect, the anti-CD20/anti-CD3 bispecific antibody, the anti-CD19/anti-CD28 bispecific antibody and the CD19-targeted 4-1BB (CD137) agonist are administered subcutaneously.

### BRIEF DESCRIPTION OF THE DRAWINGS

FIGS. 1A to 1C are schematic illustrations of a particular CD19-4-1BBL antigen binding molecules, a particular anti-CD20/anti-CD3 bispecific antibody and a particular anti-CD19/anti-CD28 bispecific antibody as used in the Examples. These molecules are described in more detail in Examples 1, 2 and 3, respectively. The thick black point stands for the knob-into-hole modification. * symbolizes amino acid modifications in the CH1 and CL domain (so-called charged variants). FIG. 1A shows a monovalent CD19 4-1BBL-trimer containing antigen binding molecule with modifications in the CH1 and CL domain adjacent to the 4-1BBL dimer and 4-1BBL monomer. The molecule is named CD19-4-1BBL herein. In FIG. 1B an exemplary bispecific anti-CD20/anti-CD3 antibody in 2+1 format is shown (named CD20-TCB or glofitamab). In FIG. 1C an exemplary bispecific anti-CD19/anti-CD28 antibody in 1+1 crossfab format is shown. The VH and VL domain of CD19 antigen binding domain are exchanged so that the VH domain is part of a light chain and the VL domain is part of a heavy chain.

FIGS. 2A to 2D show that the combination of CD20-TCB and either CD19-4-1BBL or CD19-CD28 boosts the activation of T cells as measured by the release of selected cytokines compared to CD20-TCB treatment alone in a malignant spleen resection from a stage IVB B cell lymphoma patient. Shown is the release of the cytokines Granzyme B (GzB, FIG. 2A), IFNγ (FIG. 2B), IL-8 (FIG. 2C) and IL-2 (FIG. 2D).

FIG. 3 shows the study design of an efficacy study to evaluate the triple combination effect of CD20-TCB with CD19-4-1BBL and CD19-CD28 in Human OCI-Ly18 Xenograft in humanized NSG mice. Shown is the design, i.e the various injections at different timepoints, for the different treatment groups A to G (10 mice each).

FIGS. 4A to 4G show the results of the efficacy study in OCI-Ly18 Xenograft in humanized NSG mice. Shown is the growth of tumors in individual mice for the seven treatment groups as plotted on the y-axis. FIG. 4A shows the tumor growth for each individual mouse in the vehicle group, FIG. 4B of mice treated with CD20-TCB, FIG. 4C of the mice treated with CD20-TCB and CD19-CD28 and FIG. 4D of mice treated with CD20-TCB and CD19-4-1BBL. FIG. 4E shows the tumor growth of mice that received first a treatment with the combination of CD20-TCB and CD19-4-1BBL and on day 66 the treatment was switched to the combination of CD20-TCB and CD19-CD28. FIG. 4F shows the tumor growth of the mice that received first a treatment with the combination of CD20-TCB and CD19-CD28 and afterwards (on day 66) the treatment with the combination of CD20-TCB and CD19-4-1BBL. It can be seen that the group receiving an alternating treatment regime starting with CD19-CD28 for the first four cycles followed by CD19-4-1BBL combination treatment until study termination resulted in complete tumor control over 120 days in all animals treated whereas an alternating treatment regime starting with CD19-4-1BBL for the first four cycles followed by CD19-CD28 combination treatment could not completely inhibit the tumor growth. FIG. 4G shows the tumor growth of the mice that received concomitant triple combination treatment with CD20-TCB, CD19-4-1BBL and CD19-CD28. Interestingly, the simultaneous administration of CD20-TCB, CD19-CD28 and CD19-4-1BBL did not improve the tumor growth control over the treatment with CD20-TCB and CD19-4-1BBL alone.

FIG. 5 shows the time to event analysis for the triple combination efficacy study. tumor volume cut-off of 1500 m³ was used. The probability of survival in % is plotted against the time in days. The mice that were treated first with the combination of CD20-TCB and CD19-CD28 and second with the combination of CD20-TCB and CD19-4-1BBL had the highest chance to survive (100%).

FIG. 6 shows the study design of an efficacy study to evaluate the triple combination effect of CD20-TCB with CD19-4-1BBL and CD19-CD28 in Human OCI-Ly18 Xenograft in humanized BRGS-CD47 mice. Shown is the design, i.e the various injections at different timepoints, for the different treatment groups A to G (10 mice each). The combination treatment started a week earlier compared to the first study in humanized NSG mice.

FIGS. 7A to 7G show the results of the efficacy study in OCI-Ly18 Xenograft in humanized BRGS-CD47 mice. Shown is the growth of tumors in individual mice for the seven treatment groups as plotted on the y-axis. FIG. 7A shows the tumor growth for each individual mouse in the vehicle group, FIG. 7B of mice treated with CD20-TCB only, FIG. 7C of the mice treated with CD20-TCB and CD19-CD28 and FIG. 7D of mice treated with CD20-TCB and CD19-4-1BBL. Combination treatment started on day 27. FIG. 7E shows the tumor growth of mice that received first a treatment with the combination of CD20-TCB and CD19-4-1BBL and on day 55 the treatment was switched to the combination of CD20-TCB and CD19-CD28. FIG. 7F shows the tumor growth of the mice that received first a treatment with the combination of CD20-TCB and CD19-CD28 and afterwards (on day 55) the treatment with the combination of CD20-TCB and CD19-4-1BBL. It can be seen that the group receiving an alternating treatment regime starting with CD19-CD28 for the first four cycles followed by CD19-4-1BBL combination treatment until study termination resulted in better tumor control over 94 days in most animals treated whereas an alternating treatment regime starting with CD19-4-1BBL for the first four cycles followed by CD19-CD28 combination treatment could not completely inhibit the tumor growth. FIG. 7G shows the tumor growth of the mice that received concomitant triple combination treatment with CD20-TCB, CD19-4-1BBL and CD19-CD28. The tumor control in this concomitant group is stronger than for the combination of CD20-TCB and CD19-4-1BBL in this experiment, probably due to the earlier start of combination treatment. The group receiving an alternating treatment regime starting with CD19-CD28 for the first four cycles followed by CD19-4-1BBL combination treatment until study termination (FIG. 7F) resulted in similar tumor control as the concomitant administration over 94 days.

FIGS. 8A to 8C show a comparison between the corresponding treatment regimen in both studies. Shown are the differences in tumor growth of mice that received first a treatment with the combination of CD20-TCB and CD19-4-1BBL and on day 55 a treatment with the combination of CD20-TCB and CD19-CD28 (FIG. 8A), the differences in tumor growth of the mice that received first a treatment with the combination of CD20-TCB and CD19-CD28 and afterwards (on day 55) a treatment with the combination of CD20-TCB and CD19-4-1BBL (FIG. 8B) and the differences in tumor growth of the mice that received concomitant triple combination treatment with CD20-TCB, CD19-4-1BBL and CD19-CD28 (FIG. 8C), indicating that a combination treatment starting a week earlier results in a better tumor control than a later start of the combination treatment.

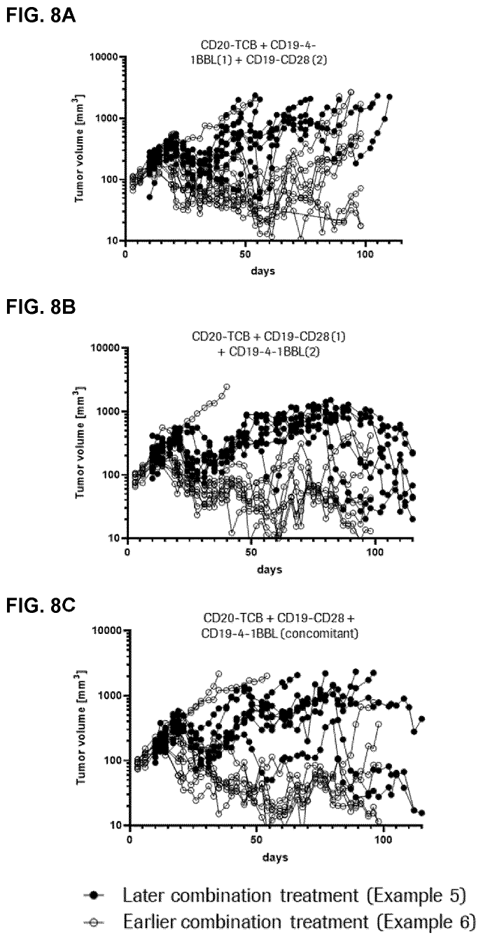

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins. The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity. The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. However, a bispecific antigen binding molecule may also comprise additional antigen binding sites which bind to further antigenic determinants. In certain aspects, the bispecific antigen binding molecule is capable of simultaneously binding two antigenic determinants, particularly two antigenic determinants expressed on two distinct cells or on the same cell. The term “bispecific” in accordance with the present disclosure thus may also include a trispecific molecule, e.g. a bispecific molecule comprising a CD28 antibody and two antigen binding domains directed to two different target cell antigens.

As used herein, the term “antigen binding domain that binds to a B cell surface antigen” or “moiety capable of specific binding to a B cell surface antigen” refers to a polypeptide molecule that specifically binds to an antigenic determinant on the B cell surface. In one aspect, the antigen binding domain is able to activate signaling through its target cell antigen. In a particular aspect, the antigen binding domain is able to direct the entity to which it is attached (e.g. the CD28 agonist) to a target site, e.g. on the B cell. Antigen binding domains capable of specific binding to a B cell surface antigen include antibodies and fragments thereof as further defined herein. In addition, antigen binding domains capable of specific binding to a B cell surface antigen include scaffold antigen binding proteins as further defined herein, e.g. binding domains which are based on designed repeat proteins or designed repeat domains (see e.g. WO 2002/020565).

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, crossFab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a variable light chain (VL) domain and a constant domain of a light chain (CL), and a variable heavy chain (VH) domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable domain (VL) and an antibody heavy chain variable domain (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “B cell surface antigen” as used herein refers to an antigenic determinant presented on the surface of a B lymphocyte, particularly a malignant B lymphocyte (in that case the antigen also being referred to as “malignant B-cell surface antigen”). Several B-cell surface antigens are interesting in terms of immunotherapy of hematologic malignant neoplasms. In one aspect, the B cell surface antigen is selected from the group consisting of CD19, CD79b, CD20, CD22 and CD37.

The term “CD19” refers to B-lymphocyte antigen CD19, also known as B-lymphocyte surface antigen B4 or T-cell surface antigen Leu-12 and includes any native CD19 from any vertebrate source, including mammals such as primates (e.g. humans) non-human primates (e.g. cynomolgus monkeys) and rodents (e.g. mice and rats), unless otherwise indicated. The amino acid sequence of human CD19 is shown in Uniprot accession no. P15391 (version 160, SEQ ID NO: 54). The term encompasses “full-length” unprocessed human CD19 as well as any form of human CD19 that results from processing in the cell as long as the antibody as reported herein binds thereto. CD19 is a structurally distinct cell surface receptor expressed on the surface of human B cells, including, but not limited to, pre-B cells, B cells in early development {i.e., immature B cells), mature B cells through terminal differentiation into plasma cells, and malignant B cells. CD19 is expressed by most pre-B acute lymphoblastic leukemias (ALL), non-Hodgkin's lymphomas, B cell chronic lymphocytic leukemias (CLL), pro-lymphocytic leukemias, hairy cell leukemias, common acute lymphocytic leukemias, and some Null-acute lymphoblastic leukemias. The expression of CD19 on plasma cells further suggests it may be expressed on differentiated B cell tumors such as multiple myeloma. Therefore, the CD19 antigen is a target for immunotherapy in the treatment of non-Hodgkin's lymphoma, chronic lymphocytic leukemia and/or acute lymphoblastic leukemia.

“CD20” refers to B-lymphocyte antigen CD20, also known as B-lymphocyte surface antigen B1 or Leukocyte surface antigen Leu-16, and includes any native CD20 from any vertebrate source, including mammals such as primates (e.g. humans) non-human primates (e.g. cynomolgus monkeys) and rodents (e.g. mice and rats), unless otherwise indicated. The amino acid sequence of human CD20 is shown in Uniprot accession no. P11836 (version 149, SEQ ID NO: 55). CD20 is a hydrophobic transmembrane protein with a molecular weight of approximately 35 kD expressed on pre-B and mature B lymphocytes. The corresponding human gene is membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein. The term “CD20” encompasses “full-length,” unprocessed CD20 as well as any form of CD20 that results from processing in the cell. The term also encompasses naturally occurring variants of CD20, e.g., splice variants or allelic variants.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³ M, e.g., from 10⁻⁹M to 10⁻¹³ M). In certain embodiments, an anti-CD20 antibody binds to an epitope of CD20 that is conserved among CD20 from different species.

By “Type II anti-CD20 antibody” is meant an anti-CD20 antibody having binding properties and biological activities of Type II anti-CD20 antibodies as described in Cragg et al., Blood 103 (2004) 2738-2743; Cragg et al., Blood 101 (2003) 1045-1052, Klein et al., mAbs 5 (2013), 22-33, and summarized in Table A below.

TABLE A
Properties of type I and type II anti-CD20 antibodies

| type I anti-CD20 antibodies | type II anti-CD20 antibodies |
|---|---|
| Bind class I CD20 epitope | Bind class II CD20 epitope |
| Localize CD20 to lipid rafts | Do not localize CD20 to lipid rafts |
| High CDC * | Low CDC * |
| ADCC activity * | ADCC activity * |
| Full binding capacity | Approx. half binding capacity |
| to B cells | to B cells |
| Weak homotypic aggregation | Homotypic aggregation |
| Low cell death induction | Strong cell death induction |

* if IgG₁ isotype

Examples of type II anti-CD20 antibodies include e.g. obinutuzumab (GA101), tositumumab (B1), humanized B-Ly1 antibody IgG1 (a chimeric humanized IgG1 antibody as disclosed in WO 2005/044859), 11B8 IgG1 (as disclosed in WO 2004/035607) and AT80 IgG1.

In one aspect, the Type II anti-CD20 antibody comprises the heavy chain variable region sequence (V_{H}CD20) of SEQ ID NO: 36 and the light chain variable region sequence (V_{L}CD20) of SEQ ID NO: 37. In another aspect, the Type II anti-CD20 antibody is engineered to have an increased proportion of non-fucosylated oligosaccharides in the Fc region as compared to a non-engineered antibody. In one aspect, at least about 40% of the N-linked oligosaccharides in the Fc region of the Type II anti-CD20 antibody are non-fucosylated. In a particular aspect, the Type II anti-CD20 antibody comprises heavy chains comprising the amino acid sequence of SEQ ID NO: 56 and light chains comprising the amino acid sequence of SEQ ID NO: 57. The antibody is named GA101 or obinutuzumab (recommended INN, WHO Drug Information, Vol. 26, No. 4, 2012, p. 453). The tradename is GAZYVA® or GAZYVARO®.

Examples of type I anti-CD20 antibodies include e.g. rituximab, ofatumumab, veltuzumab, ocaratuzumab, ocrelizumab, PRO131921, ublituximab, HI47 IgG3 (ECACC, hybridoma), 2C6 IgG1 (as disclosed in WO 2005/103081), 2F2 IgG1 (as disclosed in WO 2004/035607 and WO 2005/103081) and 2H7 IgG1 (as disclosed in WO 2004/056312).

The term “reduction” (and grammatical variations thereof such as “reduce” or “reducing”), for example reduction of the number of B cells or cytokine release, refers to a decrease in the respective quantity, as measured by appropriate methods known in the art. For clarity the term includes also reduction to zero (or below the detection limit of the analytical method), i.e. complete abolishment or elimination. Conversely, “increased” refers to an increase in the respective quantity.

A “T-cell antigen” as used herein refers to an antigenic determinant presented on the surface of a T lymphocyte, particularly a cytotoxic T lymphocyte.

A “T cell activating therapeutic agent” as used herein refers to a therapeutic agent capable of inducing T cell activation in a subject, particularly a therapeutic agent designed for inducing T-cell activation in a subject. Examples of T cell activating therapeutic agents include bispecific antibodies that specifically bind an activating T cell antigen, such as CD3, and a target cell antigen, such as CD20 or CD19. Further examples include chimeric antigen receptors (CARs) which comprise a T cell activating domain and an antigen binding moiety that specifically binds to a target cell antigen, such as CD20 or CD19.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3.

The term “CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 58. The amino acid sequence of cynomolgus [Macaca fascicularis] CD3ε is shown in NCBI GenBank no. BAB71849.1. See also SEQ ID NO: 59.

The term “CD28” (Cluster of differentiation 28, Tp44) refers to any CD28 protein from any vertebrate source, including mammals such as primates (e.g. humans) non-human primates (e.g. cynomolgus monkeys) and rodents (e.g. mice and rats), unless otherwise indicated. CD28 is expressed on T cells and provides co-stimulatory signals required for T cell activation and survival. T cell stimulation through CD28 in addition to the T-cell receptor (TCR) can provide a potent signal for the production of various interleukins. CD28 is the receptor for CD80 (B7.1) and CD86 (B7.2) proteins and is the only B7 receptor constitutively expressed on naive T cells. The amino acid sequence of human CD28 is shown in UniProt (www.uniprot.org) accession no. P10747 (SEQ ID NO:60).

An “agonistic antibody” refers to an antibody that comprises an agonistic function against a given receptor. In general, when an agonist ligand (factor) binds to a receptor, the tertiary structure of the receptor protein changes, and the receptor is activated (when the receptor is a membrane protein, a cell growth signal or such is usually transducted). If the receptor is a dimer-forming type, an agonistic antibody can dimerize the receptor at an appropriate distance and angle, thus acting similarly to a ligand. An appropriate anti-receptor antibody can mimic dimerization of receptors performed by ligands, and thus can become an agonistic antibody.

A “CD28 agonistic antibody” or “CD28 conventional agonistic antibody” is an antibody that mimicks CD28 natural ligands (CD80 or CD86) in their role to enhance T cell activation in presence of a T cell receptor signal (“signal 2”). A T cell needs two signals to become fully activated. Under physiological conditions “signal 1” arises form the interaction of T cell receptor (TCR) molecules with peptide/major histocompatibility complex (MHC) complexes on antigen presenting cells (APCs) and “signal 2” is provided by engagement of a costimulatory receptor, e.g. CD28. A CD28 agonistic antigen binding molecule is able to costimulate T cells (signal 2). It is also able to induce T cell proliferation and cytokine secretion in combination with a molecule with specificity for the TCR complex, however the CD28 agonistic antigen binding molecule is not capable of fully activating T cells without additional stimulation of the TCR. There is however a subclass of CD28 specific antigen binding molecules, the so-called CD28 superagonistic antigen binding molecules. A “CD28 superagonistic antibody” is a CD28 antibody which is capable of fully activating T cells without additional stimulation of the TCR. A CD28 superagonistic antibody is capable to induce T cell proliferation and cytokine secretion without prior T cell activation (signal 1). An example for a CD28 superagonistic antibody is TGN1412 (disclosed in WO 2006/050949).

The terms “anti-CD28 antibody”, “anti-CD28”, “CD28 antibody and “an antibody that specifically binds to CD28” refer to an antibody that is capable of binding CD28 with sufficient affinity such that the antibody is useful as a diagnostic and/or therapeutic agent in targeting CD28. In one embodiment, the extent of binding of an anti-CD28 antibody to an unrelated, non-CD28 protein is less than about 10% of the binding of the antibody to CD28 as measured, e.g., by a radioimmunoassay (RIA) or flow cytometry (FACS). In certain embodiments, an antibody that binds to CD28 has a dissociation constant (K_{D}) of ≤1 μM, ≤100 nM, ≤10 nM, ≤1 nM, ≤0.1 nM, ≤0.01 nM, or ≤0.001 nM (e.g. 10⁶ M or less, e.g. from 10⁻⁶⁸ M to 10⁻¹³ M, e.g., from 10⁻⁸ M to 10⁻¹⁰ M).

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “CH1 domain” denotes the part of an antibody heavy chain polypeptide that extends approximately from EU position 118 to EU position 215 (EU numbering system according to Kabat). In one aspect, a CH1 domain has the amino acid sequence of

(SEQ ID NO: 61)
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKV.

Usually, a segment having the amino acid sequence of EPKSC (SEQ ID NO:62) is following to link the CH1 domain to the hinge region,

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083).

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain.

The “CH2 domain” of a human IgG Fc region usually extends from an amino acid residue at about EU position 231 to an amino acid residue at about EU position 340 (EU numbering system according to Kabat). In one aspect, a CH2 domain has the amino acid sequence of APELLGGPSV FLFPPKPKDT LMISRTPEVT CVWDVSHEDP EVKENWYVDG VEVHNAKTKP REEQESTYRW SVLTVLHQDW LNGKEYKCKV SNKALPAPIE KTISKAK (SEQ ID NO: 63). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one embodiment, a carbohydrate chain is attached to the CH2 domain. The CH2 domain herein may be a native sequence CH2 domain or variant CH2 domain.

The “CH3 domain” comprises the stretch of residues C-terminal to a CH2 domain in an Fc region denotes the part of an antibody heavy chain polypeptide that extends approximately from EU position 341 to EU position 446 (EU numbering system according to Kabat). In one aspect, the CH3 domain has the amino acid sequence of GOPREPQVYT LPPSRDELTK NQVSLTCLVK GFYPSDIAVE WESNGOPENN YKTTPPVLDS DGSFFLYSKL TVDKSRWQQG NVFSCSVMHE ALHNHYTOKS LSLSPG (SEQ ID NO: 64). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof, see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “wild-type Fc domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. A human IgG1 Fc region is denoted in SEQ ID NO: 65.

The term “variant (human) Fc domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is an immune mechanism leading to lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831). For example, the capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the term “effector cells” refers to a population of lymphocytes that display effector moiety receptors, e.g. cytokine receptors, and/or Fc receptors on their surface through which they bind an effector moiety, e.g. a cytokine, and/or an Fc region of an antibody and contribute to the destruction of target cells, e.g. tumor cells. Effector cells may for example mediate cytotoxic or phagocytic effects. Effector cells include, but are not limited to, effector T cells such as CD8⁺ cytotoxic T cells, CD4⁺ helper T cells, γδ T cells, NK cells, lymphokine-activated killer (LAK) cells and macrophages/monocytes.

An “ectodomain” is the domain of a membrane protein that extends into the extracellular space (i.e. the space outside the target cell). Ectodomains are usually the parts of proteins that initiate contact with surfaces, which leads to signal transduction. The ectodomain of 4-1BBL as defined herein thus refers to the part of the 4-1BBL that extends into the extracellular space (the extracellular domain), but also includes shorter parts or fragments thereof that are responsible for the trimerization and for the binding to the corresponding receptor 4-1BB. The term “ectodomain of 4-1BBL or a fragment thereof” thus refers to the extracellular domain of 4-1BBL that forms the extracellular domain or to parts thereof that are still able to bind to the receptor (receptor binding domain).

“4-1BBL” or “4-1BB ligand” or “CD137L” is a costimulatory TNF ligand family member, which is able to costimulate proliferation and cytokine production of T-cells. Costimulatory TNF family ligands can costimulate TCR signals upon interaction with their corresponding TNF receptors and the interaction with their receptors leads to recruitment of TNFR-associated factors (TRAF), which initiate signalling cascades that result in T-cell activation. 4-1BBL is a type II transmembrane protein. Complete or full length 4-1BBL having the amino acid sequence of SEQ ID NO: 66 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO:9) form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO:4 (amino acids 52-254 of human 4-1BBL), SEQ ID NO: 1 (amino acids 71-254 of human 4-1BBL), SEQ ID NO:3 (amino acids 80-254 of human 4-1BBL), SEQ ID NO:2 (amino acids 85-254 of human 4-1BBL), SEQ ID NO:5 (amino acids 71-248 of human 4-1BBL), SEQ ID NO:6 (amino acids 85-248 of human 4-1BBL), SEQ ID NO:7 (amino acids 80-248 of human 4-1BBL), SEQ ID NO: 8 (amino acids 52-248 of human 4-1BBL) and SEQ ID NO:9 (amino acids 50-254 of human 4-1BBL), but also other fragments of the ectodomain capable of trimerization are included herein.

The term “4-1BB” or “CD137”, as used herein, refers to any native 4-1BB from any vertebrate source, including mammals such as primates (e.g. humans) and rodents (e.g., mice and rats), unless otherwise indicated. The term encompasses “full-length,” unprocessed 4-1BB as well as any form of 4-1BB that results from processing in the cell. The term also encompasses naturally occurring variants of 4-1BB, e.g., splice variants or allelic variants. The amino acid sequence of an exemplary human 4-1BB is shown in SEQ ID NO: 67 (Uniprot accession no. Q07011).

The term “peptide linker” refers to a peptide comprising one or more amino acids, typically about 2 to 20 amino acids. Peptide linkers are known in the art or are described herein. A suitable, non-immunogenic linker peptide is, for example, (G₄S)₂ (SEQ ID NO:68).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a polypeptide and an ectodomain of 4-1BBL) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 times the fraction X / Y$$

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the antigen binding molecules provided herein are contemplated. For example, it may be desirable to improve the binding affinity and/or other biological properties of the antigen binding molecules. Amino acid sequence variants of the antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table C under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g. to express the glycosyltransferase, GnTIII, or other glycosyltransferases) by the methods described herein, is relative to the ADCC mediated by the same antibody produced by the same type of non-engineered host cells.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See e.g., Grevys, A., et al., J. Immunol. 194 (2015) 5497-5508.

In another aspect, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas or lymphocytic leukemias, or melanoma.

By “B cell proliferative disorder” is meant a disease wherein the number of B cells in a patient is increased as compared to the number of B cells in a healthy subject, and particularly wherein the increase in the number of B cells is the cause or hallmark of the disease. A “CD20-positive B cell proliferative disorder” is a B cell proliferative disorder wherein B-cells, particularly malignant B-cells (in addition to normal B-cells), express CD20. Exemplary B cell proliferation disorders include Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), as well as some types of Multiple myeloma (MM) and Hodgkin lymphoma (HL). Particular B cell proliferation disorders are Non-Hodgkin lymphoma (NHL) or diffuse large B-cell lymphoma (DLBCL). In a particular aspect, the B cell proliferation disorder is diffuse large B-cell lymphoma (DLBCL).

The present invention relates to anti-CD20/anti-CD3 bispecific antibodies in combination with an anti-CD19/anti-CD28 bispecific antibody and a CD19-targeted 4-1BB (CD137) agonist for use in a combination therapy for the treatment of B-cell proliferative disorders. Scheduling studies with glofitamab and CD19-CD28 in humanized NSG mice suggested a safe and potent treatment regimen by using Gazyva pre-treatment followed by a staggered infusion of glofitmab and anti-CD19/anti-CD28 bispecific antibody CD19-CD28 applying an interval of three days at the first treatment cycle. In huNSG mice, bearing a difficult to treat disseminated WSU-DLCL2 DLBCL tumor model, the combination treatment of glofitamab and CD19-CD28 led to the formation of tumor-free animals as compared to the respective monotherapy groups. Mode of action studies in vivo revealed a strong boosting of glofitamab-mediated T cell infiltration in tumor tissue, both for CD4+ and CD8+ T cells subsets without any evidence of increased regulatory T cell activity. A second line treatment with CD19-CD28 was able to prolong the duration of glofitamab response and delayed tumor relapse in vivo in the subcutaneous OCI-Ly18 DLBCL model. Interestingly, the alternation of CD19-CD28 with a CD19-targeted 4-1BB (CD137) agonist (CD19-4-1BBL) completely prevented tumor relapse during glofitamab treatment for more than 120 days when CD19-CD28 was given for the first treatment cycles followed by CD19-4-1BBL at later cycles. Finally, CD19-CD28 boosted glofitamab-mediated cytokine secretion and T cell activation in DLBCL patient samples ex vivo validating its activity not only on T cells derived from healthy donors but also from patients. Taken together, the preclinical data show a strong rationale for combining CD19-CD28 with CD20 TCB in r/r NHL patients to deepen and further prolong the treatment response, however an optimal scheduling includes the alternating with a treatment of the combination of CD20 TCB (glofitamab) a CD19-targeted 4-1BB (CD137) agonist (CD19-4-1BBL).

### Exemplary Anti-CD20/Anti-CD3 Bispecific Antibodies for Use in the Invention

The anti-CD20/anti-CD3 bispecific antibodies as used herein are bispecific antibodies comprising a first antigen binding domain that binds to CD3, and a second antigen binding domain that binds to CD20.

Thus, the anti-CD20/anti-CD3 bispecific antibody as used herein comprises a first antigen binding domain comprising a heavy chain variable region (V_{H}CD3) and a light chain variable region (V_{L}CD3), and a second antigen binding domain comprising a heavy chain variable region (V_{H}CD20) and a light chain variable region (V_{L}CD20).

In a particular aspect, the anti-CD20/anti-CD3 bispecific antibody for use in the combination comprises a first antigen binding domain comprising a heavy chain variable region (V_{H}CD3) comprising CDR-H1 sequence of SEQ ID NO:22, CDR-H2 sequence of SEQ ID NO: 23, and CDR-H3 sequence of SEQ ID NO:24; and/or a light chain variable region (V_{L}CD3) comprising CDR-L1 sequence of SEQ ID NO:25, CDR-L2 sequence of SEQ ID NO:26, and CDR-L3 sequence of SEQ ID NO:27. More particularly, the anti-CD20/anti-CD3 bispecific comprises a first antigen binding domain comprising a heavy chain variable region (V_{H}CD3) that is at least 90%, 95%, 96%, 97%, 98%, or 99% identical to the amino acid sequence of SEQ ID NO: 28 and/or a light chain variable region (V_{L}CD3) that is at least 90%, 95%, 96%, 97%, 98%, or 99% identical to the amino acid sequence of SEQ ID NO:29. In a further aspect, the anti-CD20/anti-CD3 bispecific antibody comprises a heavy chain variable region (V_{H}CD3) comprising the amino acid sequence of SEQ ID NO:28 and/or a light chain variable region (V_{L}CD3) comprising the amino acid sequence of SEQ ID NO:29.

In one aspect, the antibody that specifically binds to CD3 is a full-length antibody. In one aspect, the antibody that specifically binds to CD3 is an antibody of the human IgG class, particularly an antibody of the human IgG1 class. In one aspect, the antibody that specifically binds to CD3 is an antibody fragment, particularly a Fab molecule or a scFv molecule, more particularly a Fab molecule. In a particular aspect, the antibody that specifically binds to CD3 is a crossover Fab molecule wherein the variable domains or the constant domains of the Fab heavy and light chain are exchanged (i.e. replaced by each other). In one aspect, the antibody that specifically binds to CD3 is a humanized antibody.

In another aspect, the anti-CD20/anti-CD3 bispecific antibody comprises a second antigen binding domain comprising a heavy chain variable region (V_{H}CD20) comprising CDR-H1 sequence of SEQ ID NO:30, CDR-H2 sequence of SEQ ID NO:31, and CDR-H3 sequence of SEQ ID NO:32, and/or a light chain variable region (V_{L}CD20) comprising CDR-L1 sequence of SEQ ID NO:33, CDR-L2 sequence of SEQ ID NO:34, and CDR-L3 sequence of SEQ ID NO:35. More particularly, the anti-CD20/anti-CD3 bispecific comprises a second antigen binding domain comprising a heavy chain variable region (V_{H}CD20) that is at least 90%, 95%, 96%, 97%, 98%, or 99% identical to the amino acid sequence of SEQ ID NO:36 and/or a light chain variable region (V_{L}CD20) that is at least 90%, 95%, 96%, 97%, 98%, or 99% identical to the amino acid sequence of SEQ ID NO:37. In a further aspect, the anti-CD20/anti-CD3 bispecific comprises a second antigen binding domain comprising a heavy chain variable region (V_{H}CD20) comprising the amino acid sequence of SEQ ID NO:36 and/or a light chain variable region (V_{L}CD20) comprising the amino acid sequence of SEQ ID NO:37.

In another particular aspect, the anti-CD20/anti-CD3 bispecific antibody comprises a third antigen binding domain that binds to CD20. In particular, the anti-CD20/anti-CD3 bispecific antibody comprises a third antigen binding domain comprising a heavy chain variable region (V_{H}CD20) comprising CDR-H1 sequence of SEQ ID NO:30, CDR-H2 sequence of SEQ ID NO: 31, and CDR-H3 sequence of SEQ ID NO:32; and/or a light chain variable region (V_{L}CD20) comprising CDR-L1 sequence of SEQ ID NO:33, CDR-L2 sequence of SEQ ID NO:34, and CDR-L3 sequence of SEQ ID NO:35. More particularly, the anti-CD20/anti-CD3 bispecific comprises a third antigen binding domain comprising a heavy chain variable region (V_{H}CD20) that is at least 90%, 95%, 96%, 97%, 98%, or 99% identical to the amino acid sequence of SEQ ID NO: 36 and/or a light chain variable region (V_{L}CD20) that is at least 90%, 95%, 96%, 97%, 98%, or 99% identical to the amino acid sequence of SEQ ID NO:37. In a further aspect, the anti-CD20/anti-CD3 bispecific comprises a third antigen binding domain comprising a heavy chain variable region (V_{H}CD20) comprising the amino acid sequence of SEQ ID NO:36 and/or a light chain variable region (V_{L}CD20) comprising the amino acid sequence of SEQ ID NO:37.

In a further aspect, the anti-CD20/anti-CD3 bispecific antibody is bispecific antibody, wherein the first antigen binding domain is a cross-Fab molecule wherein the variable domains or the constant domains of the Fab heavy and light chain are exchanged, and the second and third, if present, antigen binding domain is a conventional Fab molecule.

In another aspect, the anti-CD20/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, the (G₄S)₂ peptide linker (SEQ ID NO:68). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-CD20/anti-CD3 bispecific antibody comprises an Fc domain comprising one or more amino acid substitutions that reduce binding to an Fc receptor and/or effector function. In particular, the anti-CD20/anti-CD3 bispecific antibody comprises an IgG1 Fc domain comprising the amino acid substitutions L234A, L235A and P329G (numbering according to Kabat EU index).

In a particular aspect, the anti-CD20/anti-CD3 bispecific antibody comprises a first polypeptide comprising an amino acid sequence that is at least about 95%, 96%, 97%, 98%, 99% or 100% identical to the amino acid sequence of SEQ ID NO: 38, a second polypeptide comprising an amino acid sequence that is at least about 95%, 96%, 97%, 98%, 99% or 100% identical to the amino acid sequence of SEQ ID NO: 39, a third polypeptide comprising an amino acid sequence that is at least about 95%, 96%, 97%, 98%, 99% or 100% identical to the amino acid sequence of SEQ ID NO: 40, and a fourth and fifth polypeptide both comprising an amino acid sequence that is at least about 95%, 96%, 97%, 98%, 99% or 100% identical to the amino acid sequence of SEQ ID NO: 41. In a further particular embodiment, the bispecific antibody comprises a polypeptide sequence of SEQ ID NO: 38, a polypeptide sequence of SEQ ID NO: 39, a polypeptide sequence of SEQ ID NO: 40 and two times a polypeptide sequence of SEQ ID NO: 41 (CD20 TCB). In a particular aspect, the anti-CD20/anti-CD3 bispecific antibody is glofitamab.

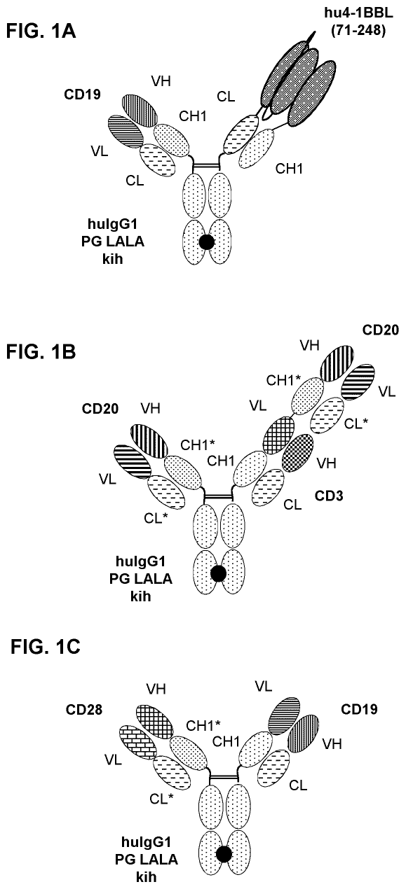

Glofitamab (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, RO7082859, or RG6026; CAS #: 2229047-91-8) is a T-cell-engaging bispecific (TCB) full-length antibody with a 2:1 molecular configuration for bivalent binding to CD20 on B cells and monovalent binding to CD3, particularly the CD3 epsilon chain (CD3e), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3e of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. A schematic picture of the molecule structure of glofitamab is depicted in FIG. 1B.

Particular other bispecific antibodies are described in PCT publication no. WO 2016/020309 A1 or in WO 2015/095392 A1. In a further aspect, the antibody is mosunetuzumab.

In a further aspect, the anti-CD20/anti-CD3 bispecific antibody may also comprise a bispecific T cell engager (BiTE®). In a further aspect, the anti-CD20/anti-CD3 bispecific antibody is XmAb®13676. In another aspect, the bispecific antibody is REGN1979. In another aspect, the bispecific antibody is FBTA05 (Lymphomun).

### Exemplary 4-1BB Agonists for Use in the Invention

In particular, the CD19-targeted 4-1BB (CD137) agonists as used in combination with the anti-CD20/anti-CD3 bispecific antibody are molecules comprising 4-1BBL. In particular, the 4-1BB agonist used in the invention comprises three ectodomains of 4-1BBL or fragments thereof.

In a particular aspect, the CD19-targeted 4-1BB (CD137) agonist is a molecule comprising three ectodomains of 4-1BBL or fragments thereof and wherein the ectodomains of 4-1BBL comprise an amino acid sequence selected from the group consisting of SEQ ID NO:1, SEQ ID NO: 2, SEQ ID NO:3, SEQ ID NO:4, SEQ ID NO:5, SEQ ID NO: 6, SEQ ID NO:7 and SEQ ID NO: 8, particularly the amino acid sequence of SEQ ID NO:5.

It has been shown that a 4-1BB agonist comprising at least one antigen binding domain capable of specific binding to CD19 was not internalized by CD19 into B cells and thus did not loss its ability to interact with the tumor microenvironment. In one aspect, provided is a 4-1BB agonist that will not be internalized in B cells, thereby maintaining its activity. In another aspect, the CD19-targeted 4-1BB (CD137) agonist is an antigen binding molecule comprising three ectodomains of 4-1BBL or fragments thereof and at least one moiety capable of specific binding to CD19, wherein the antigen binding domain capable of specific binding to CD19 is cyno-cross-reactive, i.e. the antigen binding domain capable of specific binding to CD19 specifically binds to human and to cynomolgus CD19.

In a further aspect, the CD19-targeted 4-1BB (CD137) agonist is an antigen binding molecule comprising three ectodomains of 4-1BBL or fragments thereof and at least one moiety capable of specific binding to CD19, wherein the antigen binding domain capable of specific binding to CD19 comprises a heavy chain variable region (V_{H}CD19) comprising (i) CDR-H1 comprising the amino acid sequence of SEQ ID NO: 10, (ii) CDR-H2 comprising the amino acid sequence of SEQ ID NO:11, and (iii) CDR-H3 comprising the amino acid sequence of SEQ ID NO: 12, and a light chain variable region (V_{L}CD19) comprising (iv) CDR-L1 comprising the amino acid sequence of SEQ ID NO: 13, (v) CDR-L2 comprising the amino acid sequence of SEQ ID NO:14, and (vi) CDR-L3 comprising the amino acid sequence of SEQ ID NO: 15.

In a further aspect, the CD19-targeted 4-1BB (CD137) agonist is an antigen binding molecule comprising three ectodomains of 4-1BBL or fragments thereof and at least one antigen binding domain capable of specific binding to CD19, wherein the antigen binding domain capable of specific binding to CD19 comprises a heavy chain variable region (V_{H}CD19) comprising an amino acid sequence of SEQ ID NO: 16 and a light chain variable region (V_{L}CD19) comprising an amino acid sequence of SEQ ID NO:17.

In another aspect, the CD19-targeted 4-1BB (CD137) agonist is an antigen binding molecule further comprising a Fc domain composed of a first and a second subunit capable of stable association. In one aspect, the CD19-targeted 4-1BB (CD137) agonist is an antigen binding molecule comprising an IgG Fc domain, specifically an IgG1 Fc domain or an IgG4 Fc domain. Particularly, the CD19-targeted 4-1BB (CD137) agonist is an antigen binding molecule comprising a Fc domain that comprises one or more amino acid substitution that reduces binding to an Fc receptor and/or effector function. In a particular aspect, the CD19-targeted 4-1BB (CD137) agonist is an antigen binding molecule comprising an IgG1 Fc domain comprising the amino acid substitutions L234A, L235A and P329G.

In one aspect, the CD19-targeted 4-1BB (CD137) agonist is an antigen binding molecule comprising (a) at least one antigen binding domain capable of specific binding to CD19, (b) a first and a second polypeptide that are linked to each other by a disulfide bond, wherein the first polypeptide comprises two ectodomains of 4-1BBL or fragments thereof that are connected to each other by a peptide linker and in that the second polypeptide comprises one ectodomain of 4-1BBL or a fragment thereof.

In another aspect, the CD19-targeted 4-1BB agonist comprises
(a) a first polypeptide, comprising (a1) the first ectodomain of 4-1BBL or fragment thereof, fused at its C-terminus to the N-terminus of the second ectodomain of 4-1BBL or fragment thereof, (a2) the second ectodomain of 4-1BBL or fragment thereof, fused at its C-terminus to the N-terminus of the CL domain, (a3) the CL domain, fused at its C-terminus to the N-terminus of one of the subunits (e.g. the first subunit) of the Fc domain, and (a4) one of the subunits (e.g. the first subunit) of the Fc domain;
(b) a second polypeptide, comprising (b1) the third ectodomain of 4-1BBL or fragment thereof, fused at its C-terminus to the N-terminus of the CH1 domain, and (b2) the CH1 domain;
(c) a third polypeptide, comprising (c1) the heavy chain of the Fab molecule that binds to CD19, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising the light chain of the Fab molecule that binds to CD19.

In one particular aspect, the CD19-targeted 4-1BB agonist comprises a first polypeptide comprising an amino acid sequence that is at least about 95%, 96%, 97%, 98%, 99% or 100% identical to the amino acid sequence of SEQ ID NO: 18, a second polypeptide comprising an amino acid sequence that is at least about 95%, 96%, 97%, 98%, 99% or 100% identical to the amino acid sequence of SEQ ID NO: 19, a third polypeptide comprising an amino acid sequence that is at least about 95%, 96%, 97%, 98%, 99% or 100% identical to the amino acid sequence of SEQ ID NO: 20, and a fourth polypeptide comprising an amino acid sequence that is at least about 95%, 96%, 97%, 98%, 99% or 100% identical to the amino acid sequence of SEQ ID NO: 21. More particularly, the CD19-targeted 4-1BB agonist comprises a first polypeptide comprising an amino acid sequence of SEQ ID NO: 18, a second polypeptide comprising an amino acid sequence of SEQ ID NO: 19, a third polypeptide comprising an amino acid sequence of SEQ ID NO: 20, and a fourth polypeptide comprising an amino acid sequence of SEQ ID NO: 21. In a particular aspect, the CD19-targeted 4-1BB agonist is CD19-targeted 4-1BB ligand (CD19-4-1BBL) or englumafusp alfa.

Englumafusp alfa (WHO Drug Information (International Nonproprietary Names for Pharmaceutical Substances), Recommended INN: List 89, 2023, vol. 37, no. 1, p. 97, also known as CD19-4-1BBL, RO7227166, RG6076, CAS #: 2417199-08-5) is a CD19-targeted 4-1BB ligand (CD19-4-1BBL). The underlying mode of action of this molecule is to enhance the effector function of tumor-infiltrating T-cells or NK-cells upon activation by a tumor-targeted T-cell bispecific (TCB) antibody or antibody-dependent cellular cytotoxicity (ADCC), respectively, by crosslinking 4-1BB-positive activated effector cells with CD19 positive tumor targets. Crosslinking of 4-1BB results in co-stimulation of immune cells, i.e. in enhancement of effect or functions (e.g., proliferation and production of interferon-γ and IL2, protection of cells from death (e.g., upregulation of antiapoptotic pathways genes), and promotion of immune memory development and creation of a durable immune response. By selectively promoting immune response in the microenvironment of tumors that express CD19 the risks of off-target immune responses are limited. The safety of englumafusp alfa is further increased by abolishing the interaction of Fcγ receptors (FcγRs) and the C1q complex with the IgG1 antibody portion to prevent triggering ADCC and antibody-dependent cellular phagocytosis (ADCP) by suppressing FcγR-mediated co-activation of innate immune effector cells such as NK-cells or macrophages/monocytes. A schematic picture of the molecule structure of englumafusp alfa is depicted in FIG. 1A.

In a further aspect, the 4-1BB agonist is an anti-CD19/anti-4-1BB bispecific antibody.

### Exemplary Anti-CD28 Bispecific Antibodies for Use in the Invention

The anti-CD28 bispecific antibodies as used herein are bispecific agonistic CD28 antibodies comprising an antigen binding domain capable of specific binding to CD28, an antigen binding domain capable of specific binding to a B cell surface antigen, and a Fc domain composed of a first and a second subunit capable of stable association comprising one or more amino acid substitution that reduces the binding affinity of the antigen binding molecule to an Fc receptor and/or effector function. In one aspect, the bispecific agonistic CD28 antibodies as described herein are characterized by monovalent binding to CD28. In a further aspect, the bispecific agonistic CD28 antibodies as described herein are characterized by monovalent binding to the B cell surface antigen. In particular, the B cell surface antigen is CD19.

In one aspect, a bispecific agonistic CD28 antibody as defined herein before is provided, wherein the Fc domain is an IgG, particularly an IgG1 Fc domain or an IgG4 Fc domain. In one particular aspect, the Fc domain composed of a first and a second subunit capable of stable association is an IgG1 Fc domain. The Fc domain comprises one or more amino acid substitution that reduces the binding affinity of the antigen binding molecule to an Fc receptor and/or reduces or abolishes effector function. In one aspect, the Fc domain comprises the amino acid substitutions L234A and L235A (numbering according to Kabat EU index). In one aspect, the Fc domain is of human IgG1 subclass and comprises the amino acid mutations L234A, L235A and P329G (numbering according to Kabat EU index).

In one aspect, the anti-CD19/anti-CD28 bispecific antibody as used herein comprises a first antigen binding domain comprising a heavy chain variable region (V_{H}CD28) and a light chain variable region (V_{L}CD28), and a second antigen binding domain comprising a heavy chain variable region (V_{H}CD19) and a light chain variable region (V_{L}CD19).

In a further aspect, the anti-CD19/anti-CD28 bispecific antibody comprises a first antigen binding domain comprising a heavy chain variable region (V_{H}CD28) comprising CDR-H1 sequence of SEQ ID NO:42, CDR-H2 sequence of SEQ ID NO:43, and CDR-H3 sequence of SEQ ID NO:44, and/or a light chain variable region (V_{L}CD28) comprising CDR-L1 sequence of SEQ ID NO:45, CDR-L2 sequence of SEQ ID NO:46, and CDR-L3 sequence of SEQ ID NO:47. In one further aspect, the anti-CD19/anti-CD28 bispecific antibody comprises a first antigen binding domain comprising a heavy chain variable region (V_{H}CD28) comprising the amino acid sequence that is at least about 95%, 96%, 97%, 98%, 99% or 100% identical to the amino acid sequence of SEQ ID NO:48 and/or a light chain variable region (V_{L}CD28) comprising the amino acid sequence that is at least about 95%, 96%, 97%, 98%, 99% or 100% identical to the amino acid sequence of SEQ ID NO:49. In particular, the anti-CD19/anti-CD28 bispecific antibody comprises a first antigen binding domain comprising a heavy chain variable region (V_{H}CD28) comprising the amino acid sequence of SEQ ID NO:48 and/or a light chain variable region (V_{L}CD28) comprising the amino acid sequence of SEQ ID NO:49.

In one aspect, the anti-CD19/anti-CD28 bispecific antibody comprises a second antigen binding domain comprising a heavy chain variable region (V_{H}CD19) comprising CDR-H1 sequence of SEQ ID NO:10, CDR-H2 sequence of SEQ ID NO: 11, and CDR-H3 sequence of SEQ ID NO:12; and/or a light chain variable region (V_{L}CD19) comprising CDR-L1 sequence of SEQ ID NO:13, CDR-L2 sequence of SEQ ID NO: 14, and CDR-L3 sequence of SEQ ID NO: 15. In one aspect, the anti-CD19/anti-CD28 bispecific antibody comprises a second antigen binding domain comprising a heavy chain variable region (V_{H}CD19) comprising the amino acid sequence that is at least about 95%, 96%, 97%, 98%, 99% or 100% identical to the amino acid sequence of SEQ ID NO: 16 and/or a light chain variable region (V_{L}CD19) comprising the amino acid sequence that is at least about 95%, 96%, 97%, 98%, 99% or 100% identical to the amino acid sequence of SEQ ID NO:17. In one aspect, the anti-CD19/anti-CD28 bispecific antibody comprises a second antigen binding domain comprising a heavy chain variable region (V_{H}CD19) comprising the amino acid sequence of SEQ ID NO: 16 and/or a light chain variable region (V_{L}CD19) comprising the amino acid sequence of SEQ ID NO: 17.

In one particular aspect, the anti-CD19/anti-CD28 bispecific antibody comprises a first polypeptide comprising an amino acid sequence that is at least about 95%, 96%, 97%, 98%, 99% or 100% identical to the amino acid sequence of SEQ ID NO: 50, a second polypeptide comprising an amino acid sequence that is at least about 95%, 96%, 97%, 98%, 99% or 100% identical to the amino acid sequence of SEQ ID NO: 51, a third polypeptide comprising an amino acid sequence that is at least about 95%, 96%, 97%, 98%, 99% or 100% identical to the amino acid sequence of SEQ ID NO: 52, and a fourth polypeptide comprising an amino acid sequence that is at least about 95%, 96%, 97%, 98%, 99% or 100% identical to the amino acid sequence of SEQ ID NO: 53. More particularly, the anti-CD19/anti-CD28 bispecific antibody comprises a first polypeptide comprising an amino acid sequence of SEQ ID NO: 50, a second polypeptide comprising an amino acid sequence of SEQ ID NO: 51, a third polypeptide comprising an amino acid sequence of SEQ ID NO: 52, and a fourth polypeptide comprising an amino acid sequence of SEQ ID NO: 53. A schematic picture of the molecule structure of the anti-CD19/anti-CD28 bispecific antibody is depicted in FIG. 1C.

### Preparation of Bispecific Antibodies for Use in the Invention

In certain aspects, the therapeutic agents used in the combination comprise multispecific antibodies, e.g. bispecific antibodies. Multispecific antibodies are monoclonal antibodies that have binding specificities for at least two different sites. In certain aspects, the binding specificities are for different antigens. In certain aspects, the binding specificities are for different epitopes on the same antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)), WO 93/08829, and Traunecker et al., EMBO J. 10:3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking of two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147:60 (1991).

Engineered antibodies with three or more functional antigen binding sites, including “Octopus antibodies,” are also included herein (see, e.g. US 2006/0025576A1).

The antibodies or fragments a herein also include a “Dual Acting FAb” or “DAF” comprising an antigen binding site that binds to two different antigens (see, US 2008/0069820, for example). “Crossmab” antibodies are also included herein (see e.g. WO 2009/080251, WO 2009/080252, WO2009/080253, or WO2009/080254).

Another technique for making bispecific antibody fragments is the “bispecific T cell engager” or BiTE® approach (see, e.g., WO2004/106381, WO2005/061547, WO2007/042261, and WO2008/119567). This approach utilizes two antibody variable domains arranged on a single polypeptide. For example, a single polypeptide chain includes two single chain Fv (scFv) fragments, each having a variable heavy chain (VH) and a variable light chain (VL) domain separated by a polypeptide linker of a length sufficient to allow intramolecular association between the two domains. This single polypeptide further includes a polypeptide spacer sequence between the two scFv fragments. Each scFv recognizes a different epitope, and these epitopes may be specific for different cell types, such that cells of two different cell types are brought into close proximity or tethered when each scFv is engaged with its cognate epitope. One particular embodiment of this approach includes a scFv recognizing a cell-surface antigen expressed by an immune cell, e.g., a CD3 polypeptide on a T cell, linked to another scFv that recognizes a cell-surface antigen expressed by a target cell, such as a malignant or tumor cell.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In one aspect, the bispecific bispecific antibodies used in the invention are composed of a single polypeptide chain comprising two single chain FV fragments (scFV) fused to each other by a peptide linker.

Fc Domain Modifications Reducing Fc Receptor Binding and/or Effector Function

The Fc domain of the antigen binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the antigen binding molecules of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides an antibody, wherein the Fc domain comprises one or more amino acid substitution that reduces binding to an Fc receptor, in particular towards Fcγ receptor.

In one aspect, the Fc domain of the antibody of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an antibody according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or cell activating antibodies comprising an Fc domain for Fc receptors may be evaluated using cell lines known to express particular Fc receptors, such as human NK cells expressing FcγIIIa receptor.

Effector function of an Fc domain, or antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some aspects, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antigen binding molecule of the invention is able to bind C1q and hence has CDC activity (see e.g., C1q and C3c binding ELISA in WO 2006/029879 and WO 2005/100402). To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

### Fc Domain Modifications Promoting Heterodimerization

The bispecific antigen binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antibodies of the invention in recombinant production, it will thus be advantageous to introduce in the Fc domain of the bispecific antigen binding molecules of the invention a modification promoting the association of the desired polypeptides.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in one aspect said modification is in the CH3 domain of the Fc domain.

In a specific aspect said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g. by site-specific mutagenesis, or by peptide synthesis.

In a specific such aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

The C-terminus of the heavy chain of the bispecific antibodies as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

### Modifications in the Fab Domains

In one aspect, the molecules used herein are bispecific antibodies, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange). In a particular aspect, the additional Fab fragments are Fab fragments, wherein the variable domains VL and VH are replaced by each other so that the VH domain is part of the light chain and the VL domain is part of the heavy chain.

In one aspect, the invention relates to a bispecific agonistic CD28 antigen binding molecule characterized by monovalent binding to CD28 comprising (a) one antigen binding domain capable of specific binding to CD28, (b) at least one antigen binding domain capable of specific binding to a tumor-associated antigen, and (c) a Fc domain composed of a first and a second subunit capable of stable association comprising one or more amino acid substitution that reduces the binding affinity of the antigen binding molecule to an Fc receptor and/or effector function, wherein in the Fab fragments capable of specific binding to a tumor-associated antigen the constant domains CL and CH1 are replaced by each other so that the CH1 domain is part of the light chain and the CL domain is part of the heavy chain.

In another aspect, and to further improve correct pairing, the bispecific antibodies used herein, for instance the bispecific agonistic CD28 antibodies characterized by monovalent binding to CD28 comprise (a) one Fab fragment capable of specific binding to CD28, (b) one Fab domain capable of specific binding to CD19, and (c) a Fc domain composed of a first and a second subunit capable of stable association comprising one or more amino acid substitution that reduces the binding affinity of the antigen binding molecule to an Fc receptor and/or effector function, can contain different charged amino acid substitutions (so-called “charged residues”). These modifications are introduced in the crossed or non-crossed CH1 and CL domains. In a particular aspect, the invention relates to a bispecific agonistic CD28 antigen binding molecule, wherein in one of CL domains the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E). In one particular aspect, in the CL domain of the Fab fragment capable of specific binding to CD28 the amino acid at position 123 (EU numbering) has been replaced by arginine (R) and the amino acid at position 124 (EU numbering) has been substituted by lysine (K) and in the CH1 domain of the Fab fragment capable of specific binding to CD28 the amino acids at position 147 (EU numbering) and at position 213 (EU numbering) have been substituted by glutamic acid (E).

More particularly, the bispecific used herein can comprise a Fab, wherein in the CL domain the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E).

### Pharmaceutical Compositions, Medicaments, Formulations and Routes of Administration

In a further aspect, provided are pharmaceutical compositions or medicaments comprising the anti-CD20/anti-CD3 antibodies, anti-CD19/anti-CD28 bispecific antibodies and CD19-targeted 4-1BB (CD137) agonists, e.g., for use in any of the below therapeutic methods. In one aspect, a pharmaceutical composition comprises an antibody provided herein and at least one pharmaceutically acceptable excipient. In another aspect, a pharmaceutical composition comprises an antibody provided herein and at least one additional therapeutic agent, e.g., as described below.

Pharmaceutical compositions as disclosed herein comprise a therapeutically effective amount of one or more bispecific antibodies dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one antibody and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Pharmaceutical compositions comprising the bispecific antigen binding molecules disclosed herein may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antibodies may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

### Administration of the Bispecific Antibodies

The anti-CD20/anti-CD3 bispecific antibody, the anti-CD19/anti-CD28 bispecific antibody and the CD19-targeted 4-1BB (CD137) agonist (all called substance herein) can be administered by any suitable means, including parenteral, intrapulmonary, and intranasal, and, if desired for local treatment, intralesional administration. The methods disclosed herein are particularly useful, however, in relation to therapeutic agents administered by parenteral, particularly intravenous, infusion.

Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In one aspect, the therapeutic agent is administered parenterally, particularly intravenously. In a particular aspect, the substance is administered by intravenous infusion. In another aspect, the substance is administered subcutaneously.

The anti-CD20/anti-CD3 bispecific antibody, the anti-CD19/anti-CD28 bispecific antibody and the CD19-targeted 4-1BB (CD137) agonist would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The anti-CD20/anti-CD3 bispecific antibody, the anti-CD19/anti-CD28 bispecific antibody and the CD19-targeted 4-1BB (CD137) agonist need not be, but are optionally formulated with one or more agents currently used to prevent or treat the disorder in question. The effective amount of such other agents depends on the amount of therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

The anti-CD20/anti-CD3 bispecific antibody can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. In one aspect, the anti-CD20/anti-CD3 bispecific antibody is administered parenterally, particularly intravenously, e.g. by intravenous infusion. In one aspect, the infusion rate for the anti-CD20/anti-CD3 bispecific antibody, particularly glofitamab, is at least 4 hours. In one aspect, the infusion time for the anti-CD20/anti-CD3 bispecific antibody may be reduced or extended. In one aspect, in the absence of infusion-related adverse events, the infusion time of glofitamab in subsequent cycles is reduced to 2 hours±15 minutes. In one aspect the infusion time is increased to up to 8 hours for subjects with high risk of experiencing cytokine release syndrome (CRS). In one aspect, for patients who may be at an increased risk of CRS, patients who experience IRRs or CRS with their previous dose of glofitamab or who are at increased risk of recurrent IRR/CRS with subsequent doses, the time of infusion of glofitamab is extended to up to 8 hours.

In one particular aspect, disclosed herein is the anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein, wherein the combination therapy comprises the concomitant administration of the anti-CD20/anti-CD3 bispecific antibody, the anti-CD19/anti-CD28 bispecific antibody and the CD19-targeted 4-1BB (CD137) agonist. In certain aspects, the concomitant administration is for one or more treatment cycles, particularly 3 to 8 treatment cycles. The length of a treatment cycle corresponds to 7 days or 14 days or 21 days, particularly 7 days or 14 days.

Particularly, the present disclosure also relates to the anti-CD20/anti-CD3 bispecific antibody in combination with an anti-CD19/anti-CD28 bispecific antibody and a CD19-targeted 4-1BB (CD137) agonist for use, the use, the method, the kit or the medicament as described herein, wherein the combination therapy comprises a first treatment regimen with the anti-CD20/anti-CD3 bispecific antibody in combination with an anti-CD19/anti-CD28 bispecific antibody and a second treatment regimen with the anti-CD20/anti-CD3 bispecific antibody in combination with a CD19-targeted 4-1BB (CD137) agonist.

In one aspect, the first treatment regimen with the anti-CD20/anti-CD3 bispecific antibody in combination with an anti-CD19/anti-CD28 bispecific antibody is a single administration (one treatment cycle). In certain aspects, the administration of the first treatment regimen with the anti-CD20/anti-CD3 bispecific antibody in combination with an anti-CD19/anti-CD28 bispecific antibody is two or more treatment cycles. In one aspect, the first treatment regimen comprises 1 to 5 treatment cycles and the second treatment regimen starts with the following treatment cycle. In another aspect, the first treatment regimen comprises 3 to 5 treatment cycles and the second treatment regimen starts with the following treatment cycle. In one aspect, the first treatment regimen comprises 4 treatment cycles and the second treatment regimen starts with the treatment cycle 5. In one aspect, the anti-CD19/anti-CD28 bispecific antibody is administered one hour later than the anti-CD20/anti-CD3 bispecific antibody, however in the same treatment cycle (of e.g. 7 days). In another aspect, the anti-CD19/anti-CD28 bispecific antibody is administered 2 days later than the anti-CD20/anti-CD3 bispecific antibody, however in the same treatment cycle (of e.g. 7 days or 14 days or 21 days).

In one aspect, the second treatment regimen with the anti-CD20/anti-CD3 bispecific antibody in combination with a CD19-targeted 4-1BB (CD137) agonist is a single administration (one treatment cycle). In certain aspects, the administration of the second treatment regimen with the anti-CD20/anti-CD3 bispecific antibody in combination with a CD19-targeted 4-1BB (CD137) agonist is two or more treatment cycles. In one aspect, the second treatment regimen comprises 1 to 5 treatment cycles. In another aspect, the second treatment regimen comprises 3 to 5 treatment cycles. In one aspect, the second treatment regimen comprises 2 or more treatment cycles and will be followed by repeating the first treatment regimen. In one aspect, the repeated first treatment regimen starts with the following treatment cycle. In one aspect, the repeated first treatment regimen will be followed by a repeated second treatment regimen (alternating administration).

In one such aspect, the substances are administered every week, every two weeks, or every three weeks, particularly every two weeks. In one aspect, the anti-CD20/anti-CD3 bispecific antibody is administered in a therapeutically effective amount. In one aspect the anti-CD20/anti-CD3 bispecific antibody is administered at a dose of about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg or about 1000 μg/kg. In one aspect, the anti-CD20/anti-CD3 bispecific antibody is administered at a dose which is lower than the dose of the anti-CD20/anti-CD3 bispecific antibody in a corresponding treatment regimen without the administration of the an anti-CD19/anti-CD28 bispecific antibody and a CD19-targeted 4-1BB (CD137) agonist. In one aspect, the administration of the anti-CD20/anti-CD3 bispecific antibody comprises an initial administration of a first dose of the anti-CD20/anti-CD3 bispecific antibody, and one or more subsequent administrations of a second dose of the anti-CD20/anti-CD3 bispecific antibody, wherein the second dose is higher than the first dose. In one aspect, the administration of the anti-CD20/anti-CD3 bispecific antibody comprises an initial administration of a first dose of the anti-CD20/anti-CD3 bispecific antibody, and one or more subsequent administrations of a second dose of the anti-CD20/anti-CD3 bispecific antibody, wherein the first dose is not lower than the second dose.

In one aspect, the first treatment regimen with the anti-CD20/anti-CD3 bispecific antibody in combination with an anti-CD19/anti-CD28 bispecific antibody will be started after one to three treatment cycles with single administration of the anti-CD20/anti-CD3 bispecific antibody. In one aspect, the anti-CD20/anti-CD3 bispecific antibody will be administered every week. The first treatment regimen with the anti-CD20/anti-CD3 bispecific antibody in combination with an anti-CD19/anti-CD28 bispecific antibody will be started one week after the last single administration with the anti-CD20/anti-CD3 bispecific antibody alone.

In another aspect, the administration of the anti-CD20/anti-CD3 bispecific antibody in the treatment regimen according to the invention is the first administration of the anti-CD20/anti-CD3 bispecific antibody to the subject (at least within the same course of treatment). In one aspect, no administration of the anti-CD19/anti-CD28 bispecific antibody is made to the subject prior to the administration of the anti-CD20/anti-CD3 bispecific antibody. In another aspect, the anti-CD19/anti-CD28 bispecific antibody is administered prior to the administration of the anti-CD20/anti-CD3 bispecific antibody.

In all of these aspects, the anti-CD20/anti-CD3 bispecific antibody in combination with an anti-CD19/anti-CD28 bispecific antibody and a CD19-targeted 4-1BB (CD137) agonist is for use in a combination treatment, wherein the treatment regimen starts with the administration of the anti-CD20/anti-CD3 bispecific antibody alone for one or more treatment cycles, particularly three to five treatment cycles, before the administration of the combination treatment is started. In one particular aspect, the anti-CD20/anti-CD3 bispecific antibody is administered in each single administration treatment cycle with an increasing dose (step up dosing).

In one aspect, the anti-CD20/anti-CD3 bispecific antibody is for use in combination with an anti-CD19/anti-CD28 bispecific antibody and a CD19-targeted 4-1BB (CD137) agonist, wherein a pretreatment with an Type II anti-CD20 antibody, preferably obinutuzumab, is performed prior to the combination treatment, wherein the period of time between the pretreatment and the combination treatment is sufficient for the reduction of B-cells in the individual in response to the Type II anti-CD20 antibody, preferably obinutuzumab. In one particular aspect, obinutuzumab is administered 7 days before the first treatment with the anti-CD20/anti-CD3 bispecific antibody. In one particular aspect, obinutuzumab is administered 7 days before the first treatment with the anti-CD20/anti-CD3 bispecific antibody and the anti-CD20/anti-CD3 bispecific antibody is administered on day 1 of the first treatment cycle, followed by two progressively higher dose levels of the anti-CD20/anti-CD3 bispecific antibody at day 3 and day 8 of the first treatment cycle, before the combination treatment starts with the second treatment cycle.

Activation of T cells can lead to severe cytokine release syndrome (CRS). In a phase 1 study conducted by TeGenero (Suntharalingam et al., N Engl J Med (2006) 355, 1018-1028), all 6 healthy volunteers experienced near fatal, severe cytokine release syndrome (CRS) rapidly post-infusion of an inappropriately-dosed, T-cell stimulating super-agonist anti-CD28 monoclonal antibody. The cytokine release associated with administration of a T-cell activating therapeutic agent, such as the anti-CD20/anti-CD3 bispecific antibody, to a subject can be significantly reduced by pre-treatment of said subject with a Type II anti-CD20 antibody, such as obinutuzumab, the use of GAZYVAR pre-treatment (Gpt) should aid in the rapid depletion of B cells, both in the peripheral blood and in secondary lymphoid organs, such that the risk of highly relevant adverse events (AEs) from strong systemic T cell activation by T-cell activating therapeutic agents (e.g. CRS) is reduced, while supporting exposure levels of T-cell activating therapeutic agents that are high enough from the start of dosing to mediate tumour cell elimination. To date, the safety profile of obinutuzumab (including cytokine release) has been assessed and managed in hundreds of patients in ongoing obinutuzumab clinical trials. Finally, in addition to supporting the safety profile of T-cell activating therapeutic agents such as the anti-CD20/anti-CD3 bispecific antibody, Gpt should also help prevent the formation of anti-drug antibodies (ADAs) to these unique molecules.

Such combination therapies noted above encompass combined administration (where two or more therapeutic agents are included in the same or separate formulations), and separate administration, in which case, administration of the therapeutic agent can occur prior to, simultaneously, and/or following, administration of an additional therapeutic agent or agents. In one embodiment, administration of the therapeutic agent and administration of an additional therapeutic agent occur within about one month, or within about one, two or three weeks, or within about one, two, three, four, five, or six days, of each other.

### Therapeutic Methods and Compositions

CD20 and CD19 are expressed on most B-cells (pan-B-cell marker) with the exception of stem cells and plasma cells, and are frequently expressed on most human B-cell malignancies, such as lymphoma and leukemias, e.g. in non-Hodgkin lymphoma and acute lymphoblastic leukemia. Bispecific antibodies recognizing two cell surface proteins on different cell populations hold the promise to redirect cytotoxic immune cells for destruction of pathogenic target cells.

In one aspect, there is provided a method of treating B-cell cancer in an individual in need thereof comprising administering to said individual a combination therapy comprising an anti-CD20/anti-CD3 bispecific antibody in combination with an anti-CD19/anti-CD28 bispecific antibody and an anti-CD19-targeted 4-1BB (CD137) agonist.

In one such aspect, the method further comprises administering to the subject an effective amount of at least one additional therapeutic agent. In further embodiments, herein is provided a method for depleting B-cells comprising administering to the subject an effective amount of an anti-CD20/anti-CD3 antibody and an anti-CD19/anti-CD28 bispecific antibody and/or a CD19-targeted 4-1BB (CD137) agonist. An “individual” or a “subject” according to any of the above aspects is preferably a human.

In further aspects, a composition for use in cancer immunotherapy is provided comprising an anti-CD20/anti-CD3 antibody and an anti-CD19/anti-CD28 bispecific antibody and/or a CD19-targeted 4-1BB (CD137) agonist. In certain aspects, a composition comprising an anti-CD20/anti-CD3 antibody and an anti-CD19/anti-CD28 bispecific antibody and/or a CD19-targeted 4-1BB (CD137) agonist for use in a method of cancer immunotherapy is provided.

In a further aspect, herein is provided the use of a composition comprising an anti-CD20/anti-CD3 antibody and an anti-CD19/anti-CD28 bispecific antibody and/or a CD19-targeted 4-1BB (CD137) agonist in the manufacture or preparation of a medicament. In one embodiment, the medicament is for treatment of a B-cell proliferative disorder. In a further embodiment, the medicament is for use in a method of treating a B-cell proliferative disorder comprising administering to an individual having a B-cell proliferative disorder an effective amount of the medicament. In one such embodiment, the method further comprises administering to the individual an effective amount of at least one additional therapeutic agent. In a further embodiment, the medicament is for depleting B-cells. B-cell proliferative disorders are selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), Multiple myeloma (MM) and Hodgkin lymphoma (HL). In one particular aspect, the B-cell cancer is non-Hodgkin lymphoma or diffuse large B-cell lymphoma (DLBCL).

In a further aspect, herein is provided a method for treating a B-cell cancer. In one embodiment, the method comprises administering to an individual having such B-cell cancer an effective amount of an anti-CD20/anti-CD3 bispecific antibody in combination with an anti-CD19/anti-CD28 bispecific antibody and a CD19-targeted 4-1BB (CD137) agonist. In one such embodiment, the method further comprises administering to the individual an effective amount of at least one additional therapeutic agent, as described below. An “individual” according to any of the above embodiments may be a human. The B-cell cancer is in one embodiment a B-cell lymphoma or a B-cell leukemia. In one embodiment the B-cell cancer is non-Hodgkin lymphoma or acute lymphoblastic leukemia.

The combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one embodiment, administration of the anti-CD20/anti-CD3 bispecific antibody in combination with an anti-CD19/anti-CD28 bispecific antibody and a CD19-targeted 4-1BB (CD137) agonist and administration of an additional therapeutic agent occur within about one month, or within about one, two or three weeks, or within about one, two, three, four, five, or six days, of each other.

The anti-CD20/anti-CD3 bispecific antibody in combination with an anti-CD19/anti-CD28 bispecific antibody and a CD19-targeted 4-1BB (CD137) agonist as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The anti-CD20/anti-CD3 bispecific antibody in combination with an anti-CD19/anti-CD28 bispecific antibody and a CD19-targeted 4-1BB (CD137) agonist as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

In another aspect, the anti-CD20/anti-CD3 bispecific antibody in combination with an anti-CD19/anti-CD28 bispecific antibody and a CD19-targeted 4-1BB (CD137 is for use in combination therapy, wherein a pretreatment with an Type II anti-CD20 antibody, preferably obinutuzumab, is performed prior to the combination treatment, wherein the period of time between the pretreatment and the combination treatment is sufficient for the reduction of B-cells in the individual in response to the Type II anti-CD20 antibody, preferably obinutuzumab.

Activation of T cells can lead to severe cytokine release syndrome (CRS). In a phase 1 study conducted by TeGenero (Suntharalingam et al., N Engl J Med (2006) 355, 1018-1028), all 6 healthy volunteers experienced near fatal, severe cytokine release syndrome (CRS) rapidly post-infusion of an inappropriately-dosed, T-cell stimulating super-agonist anti-CD28 monoclonal antibody. The cytokine release associated with administration of a T-cell activating therapeutic agent, such as the anti-CD20/anti-CD3 bispecific antibody, to a subject can be significantly reduced by pre-treatment of said subject with a Type II anti-CD20 antibody, such as obinutuzumab, the use of GAZYVAR pre-treatment (Gpt) should aid in the rapid depletion of B cells, both in the peripheral blood and in secondary lymphoid organs, such that the risk of highly relevant adverse events (AEs) from strong systemic T cell activation by T-cell activating therapeutic agents (e.g. CRS) is reduced, while supporting exposure levels of T-cell activating therapeutic agents that are high enough from the start of dosing to mediate tumour cell elimination. To date, the safety profile of obinutuzumab (including cytokine release) has been assessed and managed in hundreds of patients in ongoing obinutuzumab clinical trials. Finally, in addition to supporting the safety profile of T-cell activating therapeutic agents such as the anti-CD20/anti-CD3 bispecific antibody, Gpt should also help prevent the formation of anti-drug antibodies (ADAs) to these unique molecules.

### Other Agents and Treatments

The antigen binding molecules of the invention may be administered in combination with one or more other agents in therapy. For instance, a fusion protein of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent.

Such other agents are suitably present in combination in amounts that are effective for the purpose intended. The effective amount of such other agents depends on the amount of fusion protein used, the type of disorder or treatment, and other factors discussed above. The antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the antigen binding molecules of the invention can occur prior to, simultaneously, and/or following, administration of the additional therapeutic agent and/or adjuvant.

### Articles of Manufacture (Kits)

In another aspect, a kit containing materials useful for the treatment, prevention and/or diagnosis of the disorders described above is provided. The kit comprises at least one container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least three active agents in the kit are an anti-CD20/anti-CD3 bispecific antibody, an anti-CD19/anti-CD28 bispecific antibody and a CD19-targeted 4-1BB (CD137) agonist.

In a particular aspect, provided is a kit for treating or delaying progression of cancer in a subject, comprising a package comprising (A) a first composition comprising as active ingredient an anti-CD20/anti-CD3 bispecific antibody and a pharmaceutically acceptable carrier; (B) a second composition comprising as active ingredient an anti-CD19/anti-CD28 bispecific antibody and a pharmaceutically acceptable carrier, (C) a third composition comprising as active ingredient a CD19-targeted 4-1BB (CD137) agonist and a pharmaceutically acceptable carrier, and (D) instructions for using the compositions in a combination therapy.

The label or package insert indicates how the composition is used for treating the condition of choice and provides the instructions for using the compositions in a combination therapy. Moreover, the kit may comprise (a) a first container with a composition contained therein, wherein the composition comprises an anti-CD20/anti-CD3 bispecific antibody of the invention; and (b) a second container with a composition contained therein, wherein the composition comprises an anti-CD19/anti-CD28 bispecific antibody, and (c) a third container with a composition contained therein, wherein the composition comprises a CD19-targeted 4-1BB (CD137) agonist. In addition, the kit may comprise one or more further containers comprising further active ingredients that can be used in combination. The article of manufacture in this embodiment of the invention may further comprise a package insert indicating that the compositions can be used to treat a particular condition.

Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Medicaments

In another aspect, provided is a medicament comprising an anti-CD20/anti-CD3 bispecific antibody for treating B-cell proliferative disorders, wherein the anti-CD20/anti-CD3 bispecific antibody is used in combination with an anti-CD19/anti-CD28 bispecific antibody and a CD19-targeted 4-1BB (CD137) agonist.

In one aspect, provided is a medicament comprising an anti-CD20/anti-CD3 bispecific antibody for treating B-cell proliferative disorders by combined use with an anti-CD19/anti-CD28 bispecific antibody and a CD19-targeted 4-1BB (CD137) agonist. In one aspect, the anti-CD20/anti-CD3 bispecific antibody and the anti-CD19/anti-CD28 bispecific antibody and the CD19-targeted 4-1BB (CD137) agonist are administered together in a single composition or administered separately in two or more different compositions. In one particular aspect, the anti-CD20/anti-CD3 bispecific antibody and the anti-CD19/anti-CD28 bispecific antibody and the CD19-targeted 4-1BB (CD137) agonist are administered separately in three different compositions.

TABLE B
(Sequences):

| SEQ | | |
|---|---|---|
| ID NO: | Name | Sequence |
| 1 | Human (hu) 4-1BBL (71- | REGPELSPDDPAGLLDLRQGMFAQLVAQNVLLIDGPLSWY |
| | 254) | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLTQ |
| | | GATVLGLFRVTPEIPAGLPSPRSE |
| 2 | hu 4-1BBL (85-254) | LDLRQGMFAQLVAQNVLLIDGPLSWYSDPGLAGVSLTGGL |
| | | SYKEDTKELVVAKAGVYYVFFQLELRRVVAGEGSGSVSLA |
| | | LHLQPLRSAAGAAALALTVDLPPASSEARNSAFGFQGRLL |
| | | HLSAGQRLGVHLHTEARARHAWQLTQGATVLGLERVTPEI |
| | | PAGLPSPRSE |
| 3 | hu 4-1BBL (80-254) | DPAGLLDLRQGMFAQLVAQNVLLIDGPLSWYSDPGLAGVS |
| | | LTGGLSYKEDTKELVVAKAGVYYVFFQLELRRVVAGEGSG |
| | | SVSLALHLQPLRSAAGAAALALTVDLPPASSEARNSAFGF |
| | | QGRLLHLSAGQRLGVHLHTEARARHAWQLTQGATVLGLER |
| | | VTPEIPAGLPSPRSE |
| 4 | hu 4-1BBL (52-254) | PWAVSGARASPGSAASPRLREGPELSPDDPAGLLDLRQGM |
| | | FAQLVAQNVLLIDGPLSWYSDPGLAGVSLTGGLSYKEDTK |
| | | ELVVAKAGVYYVFFQLELRRVVAGEGSGSVSLALHLQPLR |
| | | SAAGAAALALTVDLPPASSEARNSAFGFQGRLLHLSAGQR |
| | | LGVHLHTEARARHAWQLTQGATVLGLFRVTPEIPAGLPSP |
| | | RSE |
| 5 | Human (hu) 4-1BBL (71- | REGPELSPDDPAGLLDLRQGMFAQLVAQNVLLIDGPLSWY |
| | 248) | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLTQ |
| | | GATVLGLFRVTPEIPAGL |
| 6 | hu 4-1BBL (85-248) | LDLRQGMFAQLVAQNVLLIDGPLSWYSDPGLAGVSLTGGL |
| | | SYKEDTKELVVAKAGVYYVFFQLELRRVVAGEGSGSVSLA |
| | | LHLQPLRSAAGAAALALTVDLPPASSEARNSAFGFQGRLL |
| | | HLSAGQRLGVHLHTEARARHAWQLTQGATVLGLERVTPEI |
| | | PAGL |
| 7 | hu 4-1BBL (80-248) | DPAGLLDLRQGMFAQLVAQNVLLIDGPLSWYSDPGLAGVS |
| | | LTGGLSYKEDTKELVVAKAGVYYVFFQLELRRVVAGEGSG |
| | | SVSLALHLQPLRSAAGAAALALTVDLPPASSEARNSAFGE |
| | | QGRLLHLSAGQRLGVHLHTEARARHAWQLTQGATVLGLER |
| | | VTPEIPAGL |
| 8 | hu 4-1BBL (52-248) | PWAVSGARASPGSAASPRLREGPELSPDDPAGLLDLRQGM |
| | | FAQLVAQNVLLIDGPLSWYSDPGLAGVSLTGGLSYKEDTK |
| | | ELVVAKAGVYYVFFQLELRRVVAGEGSGSVSLALHLQPLR |
| | | SAAGAAALALTVDLPPASSEARNSAFGFQGRLLHLSAGQR |
| | | LGVHLHTEARARHAWQLTQGATVLGLERVTPEIPAGL |
| 9 | hu 4-1BBL (50-254) | ACPWAVSGARASPGSAASPRLREGPELSPDDPAGLLDLRQ |
| | | GMFAQLVAQNVLLIDGPLSWYSDPGLAGVSLTGGLSYKED |
| | | TKELVVAKAGVYYVFFQLELRRVVAGEGSGSVSLALHLQP |
| | | LRSAAGAAALALTVDLPPASSEARNSAFGFQGRLLHLSAG |
| | | QRLGVHLHTEARARHAWQLTQGATVLGLFRVTPEIPAGLP |
| | | SPRSE |
| 10 | CD19 (8B8-2B11) CDR-H1 | DYIMH |
| 11 | CD19 (8B8-2B11) CDR-H2 | YINPYNDGSKYTEKFQG |
| 12 | CD19 (8B8-2B11) CDR-H3 | GTYYYGPQLEDY |
| 13 | CD19 (8B8-2B11) CDR-L1 | KSSQSLETSTGTTYLN |
| 14 | CD19 (8B8-2B11) CDR-L2 | RVSKRES |
| 15 | CD19 (8B8-2B11) CDR-L3 | LQLLEDPYT |
| 16 | CD19 (8B8-2B11) VH | QVQLVQSGAEVKKPGASVKVSCKASGYTFTDYIMHWVRQA |
| | | PGQGLEWMGYINPYNDGSKYTEKFQGRVTMTSDTSISTAY |
| | | MELSRLRSDDTAVYYCARGTYYYGPQLFDYWGQGTTVTVS |
| | | S |
| 17 | CD19 (8B8-2B11) VL | DIVMTQTPLSLSVTPGQPASISCKSSQSLETSTGTTYLNW |
| | | YLQKPGQSPQLLIYRVSKRFSGVPDRESGSGSGTDFTLKI |
| | | SRVEAEDVGVYYCLQLLEDPYTFGQGTKLEIK |
| 18 | hu 4-1BBL (71-248)-(G₄S)₂- | REGPELSPDDPAGLLDLRQGMFAQLVAQNVLLIDGPLSWY |
| | hu 4-1BBL (71-248)- | SDPGLAGVSLTGGLSYKEDTKELVVAKAGVYYVFFQLELR |
| | CL(RK)-Fc(knob, | RVVAGEGSGSVSLALHLQPLRSAAGAAALALTVDLPPASS |
| | PGLALA) | EARNSAFGFQGRLLHLSAGQRLGVHLHTEARARHAWQLTQ |
| | | GATVLGLFRVTPEIPAGLGGGGSGGGGSREGPELSPDDPA |
| | | GLLDLRQGMFAQLVAQNVLLIDGPLSWYSDPGLAGVSLTG |
| | | GLSYKEDTKELVVAKAGVYYVFFQLELRRVVAGEGSGSVS |
| | | LALHLQPLRSAAGAAALALTVDLPPASSEARNSAFGFQGR |
| | | LLHLSAGQRLGVHLHTEARARHAWQLTQGATVLGLFRVTP |
| | | EIPAGLGGGGSGGGGSRTVAAPSVFIFPPSDRKLKSGTAS |
| | | VVCLLNNFYPREAKVQWKVDNALQSGNSQESVTEQDSKDS |
| | | TYSLSSTLTLSKADYEKHKVYACEVTHQGLSSPVTKSENR |
| | | GECDKTHTCPPCPAPEAAGGPSVFLFPPKPKDTLMISRTP |
| | | EVTCVVVDVSHEDPEVKENWYVDGVEVHNAKTKPREEQYN |
| | | STYRVVSVLTVLHQDWLNGKEYKCKVSNKALGAPIEKTIS |
| | | KAKGQPREPQVYTLPPCRDELTKNQVSLWCLVKGFYPSDI |
| | | AVEWESNGQPENNYKTTPPVLDSDGSFFLYSKLTVDKSRW |
| | | QQGNVFSCSVMHEALHNHYTQKSLSLSP |
| 19 | hu 4-1BBL (71-248)- | REGPELSPDDPAGLLDLRQGMFAQLVAQNVLLIDGPLSWY |
| | CH1(EE) | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLTQ |
| | | GATVLGLFRVTPEIPAGLGGGGSGGGGSASTKGPSVFPLA |
| | | PSSKSTSGGTAALGCLVEDYFPEPVTVSWNSGALTSGVHT |
| | | FPAVLQSSGLYSLSSVVTVPSSSLGTQTYICNVNHKPSNT |
| | | KVDEKVEPKSC |
| 20 | CD19 (2B11) VH-CH1- | QVQLVQSGAEVKKPGASVKVSCKASGYTFTDYIMHWVRQA |
| | Fc(hole, PGLALA) | PGQGLEWMGYINPYNDGSKYTEKFQGRVTMTSDTSISTAY |
| | | MELSRLRSDDTAVYYCARGTYYYGPQLFDYWGQGTTVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKEN |
| | | WYVDGVEVHNAKTKPREEQYNSTYRVVSVLTVLHQDWLNG |
| | | KEYKCKVSNKALGAPIEKTISKAKGQPREPQVCTLPPSRD |
| | | ELTKNQVSLSCAVKGFYPSDIAVEWESNGQPENNYKTTPP |
| | | VLDSDGSFFLVSKLTVDKSRWQQGNVFSCSVMHEALHNHY |
| | | TQKSLSLSP |
| 21 | CD19 (2B11) VL-CL | DIVMTQTPLSLSVTPGQPASISCKSSQSLETSTGTTYLNW |
| | | YLQKPGQSPQLLIYRVSKRFSGVPDRFSGSGSGTDFTLKI |
| | | SRVEAEDVGVYYCLQLLEDPYTFGQGTKLEIKRTVAAPSV |
| | | FIFPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSENRGEC |
| 22 | CD3-HCDR1 | TYAMN |
| 23 | CD3-HCDR2 | RIRSKYNNYATYYADSVKG |
| 24 | CD3-HCDR3 | HGNFGNSYVSWFAY |
| 25 | CD3-LCDR1 | GSSTGAVTTSNYAN |
| 26 | CD3-LCDR2 | GTNKRAP |
| 27 | CD3-LCDR3 | ALWYSNLWV |
| 28 | CD3 VH | EVQLLESGGGLVQPGGSLRLSCAASGFTESTYAMNWVRQA |
| | | PGKGLEWVSRIRSKYNNYATYYADSVKGRFTISRDDSKNT |
| | | LYLQMNSLRAEDTAVYYCVRHGNFGNSYVSWFAYWGQGTL |
| | | VTVSS |
| 29 | CD3 VL | QAVVTQEPSLTVSPGGTVTLTCGSSTGAVTTSNYANWVQE |
| | | KPGQAFRGLIGGTNKRAPGTPARFSGSLLGGKAALTLSGA |
| | | QPEDEAEYYCALWYSNLWVFGGGTKLTVL |
| 30 | CD20-HCDR1 | YSWIN |
| 31 | CD20-HCDR2 | RIFPGDGDTDYNGKFKG |
| 32 | CD20-HCDR3 | NVFDGYWLVY |
| 33 | CD20-LCDR1 | RSSKSLLHSNGITYLY |
| 34 | CD20-LCDR2 | QMSNLVS |
| 35 | CD20-LCDR3 | AQNLELPYT |
| 36 | CD20 VH | QVQLVQSGAEVKKPGSSVKVSCKASGYAFSYSWINWVRQA |
| | | PGQGLEWMGRIFPGDGDTDYNGKFKGRVTITADKSTSTAY |
| | | MELSSLRSEDTAVYYCARNVEDGYWLVYWGQGTLVTVSS |
| 37 | CD20 VL | DIVMTQTPLSLPVTPGEPASISCRSSKSLLHSNGITYLYW |
| | | YLQKPGQSPQLLIYQMSNLVSGVPDRESGSGSGTDETLKI |
| | | SRVEAEDVGVYYCAQNLELPYTFGGGTKVEIK |
| 38 | CD20 VH-CH1(EE)-CD3 | QVQLVQSGAEVKKPGSSVKVSCKASGYAFSYSWINWVRQA |
| | VL-CH1-Fc (knob, P329G | PGQGLEWMGRIFPGDGDTDYNGKFKGRVTITADKSTSTAY |
| | LALA) | MELSSLRSEDTAVYYCARNVEDGYWLVYWGQGTLVTVSSA |
| | | STKGPSVFPLAPSSKSTSGGTAALGCLVEDYFPEPVTVSW |
| | | NSGALTSGVHTFPAVLQSSGLYSLSSVVTVPSSSLGTQTY |
| | | ICNVNHKPSNTKVDEKVEPKSCDGGGGSGGGGSQAVVTQE |
| | | PSLTVSPGGTVTLTCGSSTGAVTTSNYANWVQEKPGQAFR |
| | | GLIGGTNKRAPGTPARFSGSLLGGKAALTLSGAQPEDEAE |
| | | YYCALWYSNLWVFGGGTKLTVLSSASTKGPSVFPLAPSSK |
| | | STSGGTAALGCLVKDYFPEPVTVSWNSGALTSGVHTFPAV |
| | | LQSSGLYSLSSVVTVPSSSLGTQTYICNVNHKPSNTKVDK |
| | | KVEPKSCDKTHTCPPCPAPEAAGGPSVFLFPPKPKDTLMI |
| | | SRTPEVTCVVVDVSHEDPEVKENWYVDGVEVHNAKTKPRE |
| | | EQYNSTYRVVSVLTVLHQDWLNGKEYKCKVSNKALGAPIE |
| | | KTISKAKGQPREPQVYTLPPCRDELTKNQVSLWCLVKGFY |
| | | PSDIAVEWESNGQPENNYKTTPPVLDSDGSFFLYSKLTVD |
| | | KSRWQQGNVFSCSVMHEALHNHYTQKSLSLSP |
| 39 | CD20 VH-CH1(EE)-Fc | QVQLVQSGAEVKKPGSSVKVSCKASGYAFSYSWINWVRQA |
| | (hole, P329G LALA) | PGQGLEWMGRIFPGDGDTDYNGKFKGRVTITADKSTSTAY |
| | | MELSSLRSEDTAVYYCARNVEDGYWLVYWGQGTLVTVSSA |
| | | STKGPSVFPLAPSSKSTSGGTAALGCLVEDYFPEPVTVSW |
| | | NSGALTSGVHTFPAVLQSSGLYSLSSVVTVPSSSLGTQTY |
| | | ICNVNHKPSNTKVDEKVEPKSCDKTHTCPPCPAPEAAGGP |
| | | SVFLFPPKPKDTLMISRTPEVTCVVVDVSHEDPEVKENWY |
| | | VDGVEVHNAKTKPREEQYNSTYRVVSVLTVLHQDWLNGKE |
| | | YKCKVSNKALGAPIEKTISKAKGQPREPQVCTLPPSRDEL |
| | | TKNQVSLSCAVKGFYPSDIAVEWESNGQPENNYKTTPPVL |
| | | DSDGSFFLVSKLTVDKSRWQQGNVFSCSVMHEALHNHYTQ |
| | | KSLSLSP |
| 40 | CD3 VH-CL | EVQLLESGGGLVQPGGSLRLSCAASGFTFSTYAMNWVRQA |
| | | PGKGLEWVSRIRSKYNNYATYYADSVKGRFTISRDDSKNT |
| | | LYLQMNSLRAEDTAVYYCVRHGNFGNSYVSWFAYWGQGTL |
| | | VTVSSASVAAPSVFIFPPSDEQLKSGTASVVCLLNNFYPR |
| | | EAKVQWKVDNALQSGNSQESVTEQDSKDSTYSLSSTLTLS |
| | | KADYEKHKVYACEVTHQGLSSPVTKSENRGEC |
| 41 | CD20 VL-CL(RK) | DIVMTQTPLSLPVTPGEPASISCRSSKSLLHSNGITYLYW |
| | | YLQKPGQSPQLLIYQMSNLVSGVPDRFSGSGSGTDFTLKI |
| | | SRVEAEDVGVYYCAQNLELPYTFGGGTKVEIKRTVAAPSV |
| | | FIFPPSDRKLKSGTASVVCLLNNFYPREAKVQWKVDNALQ |
| | | SGNSQESVTEQDSKDSTYSLSSTLTLSKADYEKHKVYACE |
| | | VTHQGLSSPVTKSENRGEC |
| 42 | CD28-HCDR1 | SYYIH |
| 43 | CD28-HCDR2 | SIYPGNVQTNYNEKFKD |
| 44 | CD28-HCDR3 | SHYGLDENEDV |
| 45 | CD28-LCDR1 | HASQNIYVYLN |
| 46 | CD28-LCDR2 | KASNLHT |
| 47 | CD28-LCDR3 | QQGQTYPYT |
| 48 | CD28 (SA_v8) VH | QVQLVQSGAEVKKPGASVKVSCKASGYTFTSYYIHWVRQA |
| | | PGQGLEWIGSIYPGNVQTNYNEKFKDRATLTVDTSISTAY |
| | | MELSRLRSDDTAVYFCTRSHYGLDENFDVWGQGTTVTVSS |
| 49 | CD28 (SA_v8) VL | DIQMTQSPSSLSASVGDRVTITCHASQNIYVYLNWYQQKP |
| | | GKAPKLLIYKASNLHTGVPSRFSGSGSGTDFTLTISSLQP |
| | | EDFATYYCQQGQTYPYTFGGGTKVEIK |
| 50 | CD28 heavy chain | QVQLVQSGAEVKKPGASVKVSCKASGYTFTSYYIHWVRQA |
| | | PGQGLEWIGSIYPGNVQTNYNEKFKDRATLTVDTSISTAY |
| | | MELSRLRSDDTAVYFCTRSHYGLDFNFDVWGQGTTVTVSS |
| | | ASTKGPSVFPLAPSSKSTSGGTAALGCLVEDYFPEPVTVS |
| | | WNSGALTSGVHTFPAVLQSSGLYSLSSVVTVPSSSLGTQT |
| | | YICNVNHKPSNTKVDEKVEPKSCDKTHTCPPCPAPEAAGG |
| | | PSVFLFPPKPKDTLMISRTPEVTCVVVDVSHEDPEVKENW |
| | | YVDGVEVHNAKTKPREEQYNSTYRVVSVLTVLHQDWLNGK |
| | | EYKCKVSNKALGAPIEKTISKAKGQPREPQVYTLPPCRDE |
| | | LTKNQVSLWCLVKGFYPSDIAVEWESNGQPENNYKTTPPV |
| | | LDSDGSFFLYSKLTVDKSRWQQGNVFSCSVMHEALHNHYT |
| | | QKSLSLSP |
| 51 | CD28 light chain | DIQMTQSPSSLSASVGDRVTITCHASQNIYVYLNWYQQKP |
| | | GKAPKLLIYKASNLHTGVPSRFSGSGSGTDETLTISSLQP |
| | | EDFATYYCQQGQTYPYTFGGGTKVEIKRTVAAPSVFIFPP |
| | | SDRKLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSENRGEC |
| 52 | CD19 heavy chain | DIVMTQTPLSLSVTPGQPASISCKSSQSLETSTGTTYLNW |
| | | YLQKPGQSPQLLIYRVSKRFSGVPDRFSGSGSGTDFTLKI |
| | | SRVEAEDVGVYYCLQLLEDPYTFGQGTKLEIKSSASTKGP |
| | | SVFPLAPSSKSTSGGTAALGCLVKDYFPEPVTVSWNSGAL |
| | | TSGVHTFPAVLQSSGLYSLSSVVTVPSSSLGTQTYICNVN |
| | | HKPSNTKVDKKVEPKSCDKTHTCPPCPAPEAAGGPSVFLF |
| | | PPKPKDTLMISRTPEVTCVVVDVSHEDPEVKENWYVDGVE |
| | | VHNAKTKPREEQYNSTYRVVSVLTVLHQDWLNGKEYKCKV |
| | | SNKALGAPIEKTISKAKGQPREPQVCTLPPSRDELTKNQV |
| | | SLSCAVKGFYPSDIAVEWESNGQPENNYKTTPPVLDSDGS |
| | | FFLVSKLTVDKSRWQQGNVFSCSVMHEALHNHYTQKSLSL |
| | | SP |
| 53 | CD19 light chain | QVQLVQSGAEVKKPGASVKVSCKASGYTFTDYIMHWVRQA |
| | | PGQGLEWMGYINPYNDGSKYTEKFQGRVTMTSDTSISTAY |
| | | MELSRLRSDDTAVYYCARGTYYYGPQLFDYWGQGTTVTVS |
| | | SASVAAPSVFIFPPSDEQLKSGTASVVCLLNNFYPREAKV |
| | | QWKVDNALQSGNSQESVTEQDSKDSTYSLSSTLTLSKADY |
| | | EKHKVYACEVTHQGLSSPVTKSENRGEC |
| 54 | human CD19 (Uniprot | MPPPRLLFFLLFLTPMEVRPEEPLVVKVEEGDNAVLQCLKGTSD |
| | Accession No. P15391) | GPTQQLTWSRESPLKPFLKLSLGLPGLGIHMRPLAIWLFIENVS |
| | | QQMGGFYLCQPGPPSEKAWQPGWTVNVEGSGELFRWNVSDLGGL |
| | | GCGLKNRSSEGPSSPSGKLMSPKLYVWAKDRPEIWEGEPPCLPP |
| | | RDSLNQSLSQDLTMAPGSTLWLSCGVPPDSVSRGPLSWTHVHPK |
| | | GPKSLLSLELKDDRPARDMWVMETGLLLPRATAQDAGKYYCHRG |
| | | NLTMSFHLEITARPVLWHWLLRTGGWKVSAVTLAYLIFCLCSLV |
| | | GILHLQRALVLRRKRKRMTDPTRRFFKVTPPPGSGPQNQYGNVL |
| | | SLPTPTSGLGRAQRWAAGLGGTAPSYGNPSSDVQADGALGSRSP |
| | | PGVGPEEEEGEGYEEPDSEEDSEFYENDSNLGQDQLSQDGSGYE |
| | | NPEDEPLGPEDEDSFSNAESYENEDEELTQPVARTMDFLSPHGS |
| | | AWDPSREATSLAGSQSYEDMRGILYAAPQLRSIRGQPGPNHEED |
| | | ADSYENMDNPDGPDPAWGGGGRMGTWSTR |
| 55 | Human CD20 (Uniprot | MTTPRNSVNGTFPAEPMKGPIAMQSGPKPLFRRMSSLVGPTQSF |
| | Accession No. P11836) | FMRESKTLGAVQIMNGLFHIALGGLLMIPAGIYAPICVTVWYPL |
| | | WGGIMYIISGSLLAATEKNSRKCLVKGKMIMNSLSLFAAISGMI |
| | | LSIMDILNIKISHFLKMESLNFIRAHTPYINIYNCEPANPSEKN |
| | | SPSTQYCYSIQSLFLGILSVMLIFAFFQELVIAGIVENEWKRTC |
| | | SRPKSNIVLLSAEEKKEQTIEIKEEVVGLTETSSQPKNEEDIEI |
| | | IPIQEEEEEETETNFPEPPQDQESSPIENDSS |
| 56 | Obinutuzumab heavy chain | QVQLVQSGAEVKKPGSSVKVSCKASGYAFSYSWINWVRQA |
| | | PGQGLEWMGRIFPGDGDTDYNGKFKGRVTITADKSTSTAY |
| | | MELSSLRSEDTAVYYCARNVEDGYWLVYWGQGTLVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSCDKTHTCPPCPAPELLGGP |
| | | SVFLFPPKPKDTLMISRTPEVTCVVVDVSHEDPEVKENWY |
| | | VDGVEVHNAKTKPREEQYNSTYRVVSVLTVLHQDWLNGKE |
| | | YKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSP |
| 57 | Obinutuzumab light chain | DIVMTQTPLSLPVTPGEPASISCRSSKSLLHSNGITYLYW |
| | | YLQKPGQSPQLLIYQMSNLVSGVPDRESGSGSGTDFTLKI |
| | | SRVEAEDVGVYYCAQNLELPYTFGGGTKVEIKRTVAAPSV |
| | | FIFPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSENRGEC |
| 58 | human CD3ϵ (UniProt | MQSGTHWRVLGLCLLSVGVWGQDGNEEMGGITQTPYKVSISGTT |
| | accession no. P07766) | VILTCPQYPGSEILWQHNDKNIGGDEDDKNIGSDEDHLSLKEFS |
| | | ELEQSGYYVCYPRGSKPEDANFYLYLRARVCENCMEMDVMSVAT |
| | | IVIVDICITGGLLLLVYYWSKNRKAKAKPVTRGAGAGGRQRGQN |
| | | KERPPPVPNPDYEPIRKGQRDLYSGLNQRRI |
| 59 | cynomolgus CD3ϵ (NCBI | MQSGTRWRVLGLCLLSIGVWGQDGNEEMGSITQTPYQVSI |
| | GenBank no. BAB71849.1) | SGTTVILTCSQHLGSEAQWQHNGKNKEDSGDRLFLPEFSE |
| | | MEQSGYYVCYPRGSNPEDASHHLYLKARVCENCMEMDVMA |
| | | VATIVIVDICITLGLLLLVYYWSKNRKAKAKPVTRGAGAG |
| | | GRQRGQNKERPPPVPNPDYEPIRKGQQDLYSGLNQRRI |
| 60 | human CD28 (Uniprot | MLRLLLALNLFPSIQVTGNKILVKQSPMLVAYDNAVNLSCKYSY |
| | accession No. P10747) | NLFSREFRASLHKGLDSAVEVCVVYGNYSQQLQVYSKTGENCDG |
| | | KLGNESVTFYLQNLYVNQTDIYFCKIEVMYPPPYLDNEKSNGTI |
| | | IHVKGKHLCPSPLFPGPSKPFWVLVVVGGVLACYSLLVTVAFII |
| | | FWVRSKRSRLLHSDYMNMTPRRPGPTRKHYQPYAPPRDFAAYRS |
| 61 | CH1 Domain | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKV |
| 62 | Hinge connection | EPKSC |
| 63 | CH2 domain | APELLGGPSVFLFPPKPKDTLMISRTPEVTCVWDVSHEDP |
| | | EVKFNWYVDGVEVHNAKTKPREEQESTYRWSVLTVLHQDW |
| | | LNGKEYKCKVSNKALPAPIEKTISKAK |
| 64 | CH3 domain | GQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPG |
| 65 | Hu IgG1 Fc region | DKTHTCPPCPAPELLGGPSVELFPPKPKDTLMISRTPEVT |
| | | CVVVDVSHEDPEVKENWYVDGVEVHNAKTKPREEQYNSTY |
| | | RVVSVLTVLHQDWLNGKEYKCKVSNKALPAPIEKTISKAK |
| | | GQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSP |
| 66 | Full length 4-1BBL | MEYASDASLDPEAPWPPAPRARACRVLPWALVAGLLLLLLLAAA |
| | (UniProt No. P41273) | CAVFLACPWAVSGARASPGSAASPRLREGPELSPDDPAGLLDLR |
| | | QGMFAQLVAQNVLLIDGPLSWYSDPGLAGVSLTGGLSYKEDTKE |
| | | LVVAKAGVYYVFFQLELRRVVAGEGSGSVSLALHLQPLRSAAGA |
| | | AALALTVDLPPASSEARNSAFGFQGRLLHLSAGQRLGVHLHTEA |
| | | RARHAWQLTQGATVLGLFRVTPEIPAGLPSPRSE |
| 67 | Human 4-1BB (Uniprot | MGNSCYNIVATLLLVLNFERTRSLQDPCSNCPAGTFCDNNRNQI |
| | accession No. Q07011) | CSPCPPNSFSSAGGQRTCDICRQCKGVFRTRKECSSTSNAECDC |
| | | TPGFHCLGAGCSMCEQDCKQGQELTKKGCKDCCFGTENDQKRGI |
| | | CRPWTNCSLDGKSVLVNGTKERDVVCGPSPADLSPGASSVTPPA |
| | | PAREPGHSPQIISFFLALTSTALLFLLFFLTLRFSVVKRGRKKL |
| | | LYIFKQPFMRPVQTTQEEDGCSCRFPEEEEGGCEL |
| 68 | Peptide linker | GGGGSGGGGS |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, MD (1991)) as defined above.

### EXAMPLES

The following are examples of methods and compositions of the invention. It is understood that various other embodiments may be practiced, given the general description provided above.

### Example 1

### Preparation, Purification and Characterization of CD19-41BBL Antigen Binding Molecules

CD19-targeted 4-1BB ligand trimer-containing Fc fusion antigen binding molecules were prepared as described in International Patent Appl. Publ. No. WO 2016/075278 A1, in particular Example 7.2.7 (Construct 4.5).

For the preparation, a polypeptide including a dimeric 4-1BB ligand fused to human CL domain was subcloned in frame with the human IgG1 heavy chain CH2 and CH3 domains on the knob. A polypeptide including one ectodomain of the 4-1BB ligand was fused to the human IgG1-CH1 domain. In order to improve correct pairing amino acid mutations were additionally introduced in the crossed CH-CL (charged variants), in the CL domain E123R and Q124K and in the CH1 domain K147E and K213E (EU numbering according to Kabat).

The variable region of heavy and light chain DNA sequences encoding CD19 antibody clone 8B8-2B11, were subcloned in frame with either the constant heavy chain of the hole or the constant light chain of human IgG1. The Pro329Gly, Leu234Ala and Leu235Ala mutations were introduced in the constant region of the knob and hole heavy chains to abrogate binding to Fc gamma receptors according to the method described in WO 2012/130831. Combination of the dimeric ligand-Fc knob chain containing the S354C/T366W mutations, the monomeric CH1 fusion, the targeted anti-CD19-Fc hole chain containing the Y349C/T366S/L368A/Y407V mutations and the anti-CD19 light chain allowed the generation of a heterodimer, which includes an assembled trimeric 4-1BB ligand and a CD19 binding Fab. The molecule is called herein CD19-4-1BBL.

CD19-4-1BBL comprises the amino acid sequences of SEQ ID NO:57, SEQ ID NO:58, SEQ ID NO:59 and SEQ ID NO:60. A schematic scheme of the CD19-targeted 4-1BB ligand trimer-containing Fc fusion antigen binding molecule is shown in FIG. 1A.

The production and characterization of CD19-targeted and untargeted 4-1BB ligand trimer-containing Fc fusion antigen binding molecules is described in detail in WO 2016/075278, Example 7.4 and Examples 8 to 11, respectively.

### Example 2

### Preparation, Purification and Characterization of T-Cell Bispecific (TCB) Antibodies

TCB molecules have been prepared according to the methods described in WO 2016/020309 A1.

The anti-CD20/anti-CD3 bispecific antibody (CD20 CD3 TCB or CD20 TCB or glofitamab) used in the experiments corresponds to molecule B as described in Example 1 of WO 2016/020309 A1. Molecule B is a “2+1 IgG CrossFab” antibody and is comprised of two different heavy chains and two different light chains. Point mutations in the CH3 domain (“knobs into holes”) were introduced to promote the assembly of the two different heavy chains. The Pro329Gly, Leu234Ala and Leu235Ala mutations were introduced in the constant region of the knob and hole heavy chains to abrogate binding to Fc gamma receptors according to the method described in WO 2012/130831. Exchange of the VH and VL domains in the CD3 binding Fab and point mutations in the CH and CL domains in the CD20 binding Fab were made in order to promote the correct assembly of the two different light chains. 2+1 means that the molecule has two antigen binding domains specific for CD20 and one antigen binding domain specific for CD3.

CD20 TCB comprises the amino acid sequences of SEQ ID NO:57, SEQ ID NO:58, SEQ ID NO: 59 and SEQ ID NO:60. A schematic scheme of the bispecific antibody in 2+1 format is shown in FIG. 1B.

The molecule is further characterized in Example 1 of WO 2016/020309 A1.

### Example 3

### Preparation, Purification and Characterization of CD19-CD28 Bispecific Antibodies

CD19-CD28 bispecific antibodies were prepared as described in International Patent Appl. Publ. No. WO 2020/127618 A1.

More particularly, the generation and production is described in Example 18. For the generation of the respective expression plasmids, the sequences of the variable domains of CD19 antibody clone 8B8-2B11 and CD28 antibody clone SA_v8 were sub-cloned in frame with the respective constant regions which are pre-inserted in the respective recipient mammalian expression vector. A schematic description of the resulting molecule is shown in FIG. 1C. It is a “1+1 IgG1 CrossFab” antibody and is comprised of two different heavy chains and two different light chains. Point mutations in the CH3 domain (“knobs into holes”) were introduced to promote the assembly of the two different heavy chains. The Pro329Gly, Leu234Ala and Leu235 Ala mutations were introduced in the constant region of the knob and hole heavy chains to abrogate binding to Fc gamma receptors according to the method described in WO 2012/130831. Exchange of the VH and VL domains in the CD19 binding Fab and point mutations in the CH and CL domains in the CD28 binding Fab were made in order to promote the correct assembly of the two different light chains.

CD20 TCB comprises the amino acid sequences of SEQ ID NO:57, SEQ ID NO:58, SEQ ID NO: 59 and SEQ ID NO:60.

### Example 4

### Combination Therapy of CD19-CD28, CD19-4-1BBL and CD20 TCB Ex Vivo

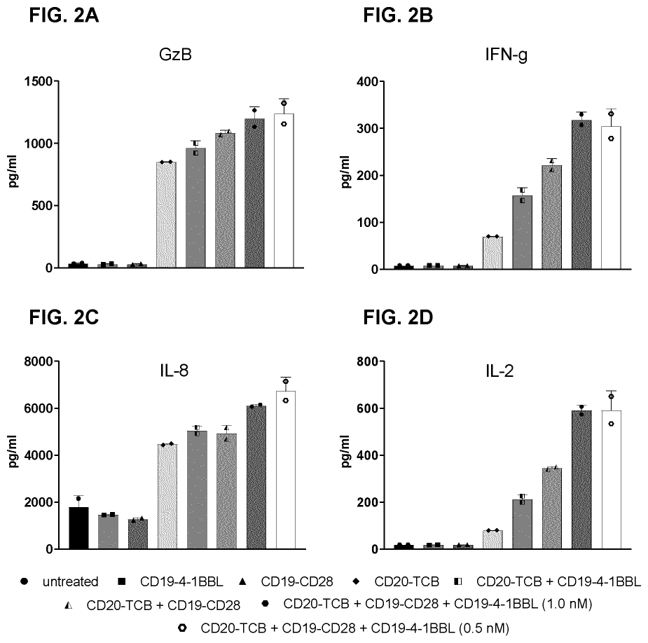

Our hypothesis is that CD19-CD28 as well as CD19-4-1BBL can synergize with CD20-TCB for T cell activation. To test this, we digested a malignant spleen resection from a stage IVB B cell lymphoma patient and incubated the cells with either CD20-TCB (25 μM) or one of the two costimulators CD19-CD28 or CD19-4-1BBL (1 nM) alone or combinations of the TCB and the costimulators. After 3 days, cytokine release in the supernatants was determined using CBA Kits (Cytometric Bead Array, BD Biosciences). FIGS. 2A to 2D show that CD20-TCB induces cytokine release (GzB, IFNg, IL-2 and IL-8) and that CD19-CD28 as well as CD19-4-1BBL could further boost the CD20-TCB-induced cytokine release (especially IFNg and IL-2, see FIGS. 2B and 2D, respectively). The triple combination of CD20-TCB and both costimulators (using either 0.5 nM or 1 nM of each costimulator) showed further increased cytokine release (especially IFNg and IL-2) compared to the double combinations of CD20-TCB and either CD19-CD28 or CD19-4-1BBL.

### Example 5

Efficacy Study to Evaluate the Triple Combination Effect of CD20-TCB with CD19-4-1BBL and CD19-CD28 in Humanized NSG Mice

The efficacy study described in here aimed to evaluate a potential triple combination effect of CD20-TCB with CD19-CD28 and CD19-41BBL in a CD19/CD20-positive human lymphoma model in fully humanized NSG mice.

Human OCI-Ly18 (Diffuse large B cell lymphoma; DLBCL) were originally obtained from ATCC and after expansion deposited in the Roche Glycart internal cell bank. Cells were cultured in RPMI containing 10% FCS and 1× Glutamax. The cells were cultured at 37° C. in a water-saturated atmosphere at 5% CO₂. 50 microliters cell suspension (5×10⁶ NALM6 cells) mixed with 50 microliters Matrigel were injected subcutaneously in the flank of anaesthetized mice with a 22G to 30G needle.

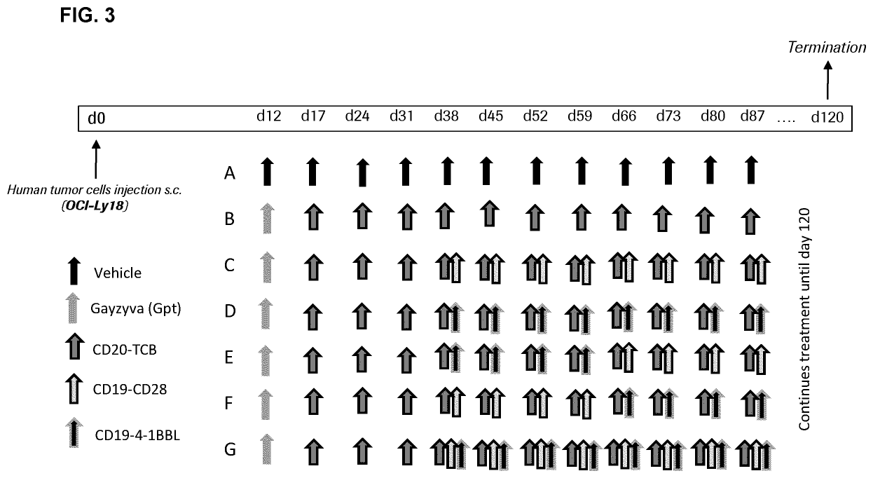

Female NSG mice, at the age 4-5 weeks at start of the experiment (bred at Jackson Laboratory) were maintained under specific-pathogen-free condition with daily cycles of 12 h light/12 h darkness according to committed guidelines (GV-Solas; Felasa; TierschG). The experimental study protocol was reviewed and approved by local government (ZH183/2020). After arrival, animals were maintained for one week to get accustomed to the new environment and for observation. Continuous health monitoring was carried out on a regular basis. Mice were injected i.p. with 15 mg/kg of Busulfan followed one day later by an i.v. injection of 1×105 human hematopoietic stem cells isolated from cord blood. At week 14-16 after stem cell injection mice were bled sublingual and blood was analyzed by flow cytometry for successful humanization. Efficiently engrafted mice were randomized according to their human T cell frequencies into the different treatment groups. At that time, mice were injected with tumor cells s.c. as described (FIG. 3) and treated with the compounds or Histidine buffer (Vehicle; Group A) when tumor size reached appr. 250 mm³ (at day 12). All mice were injected i.v. with 200 μl of the appropriate solution. To obtain the proper amount of compounds per 200 μl, the stock solutions (Table 1) were diluted with Histidine buffer when necessary. For combination treatments (Groups C to G), antibodies were mixed and injected concomitantly. All combination treatments started at day 38 upon tumor cell injection after receiving GAZYVA and three cycles of CD20-TCB. As displayed in FIG. 3, Group C received a combination with CD19-CD28 and Group D with CD19-4-1BBL. Group G received a triple combination injected concomitantly from day 38 onwards. Groups E and F received an alternating treatment schedule where treatment was either started with CD19-CD28 (Group F) or with CD19-4-1BBL (Group E) for four cycles followed by the injection of the other costimulatory molecule for the rest of all treatment cycles.

TABLE 1
Compositions used in this experiment

| | Formulation | Concentration |
|---|---|---|
| Compound | buffer | (mg/mL) |
| GAYZYVA | 20 mM Histidine, | 25.8 |
| (ID: P1AD3543-006) | 140 mM NaCl, | (=stock |
| | pH 6.0 | solution) |
| CD20-TCB | 20 mM Histidine, | 19.7 |
| (ID: P1AA4006-37290) | 140 mM NaCl, | (=stock |
| | pH 6.0 | solution) |
| CD19-CD28 | 20 mM Histidine, | 26.11 |
| (ID: P1AF0175-38963) | 140 mM NaCl, | (=stock |
| | pH 6.0 | solution) |
| CD19-4-1BBL | 20 mM Histidine, | 23.9 |
| (ID: P1AA0727-120602) | 140 mM NaCl, | (=stock |
| | pH 6.0 | solution) |

TABLE 2
Groups and their treatment regimen

| | No. of | | Dose | Injection | No. of |
|---|---|---|---|---|---|
| Group | animals | Compound | (mg/kg) | route | treatments |
| A | 10 | Vehicle | — | i.v. | 15 (once weekly) |
| B | 10 | Gpt + CD20-TCB | 30/5 | i.v. | 1 (once) + |
| | | | | | 14 (once weekly) |
| C | 10 | Gpt + CD20-TCB + | 30/5/1 | i.v. | 1 (once) + |
| | | CD19-CD28 | | | 14 (once weekly) + |
| | | | | | 11 (once weekly) |
| D | 10 | Gpt + CD20-TCB + | 30/5/1 | i.v. | 1 (once) + |
| | | CD19-4-1BBL | | | 14 (once weekly) + |
| | | | | | 11 (once weekly) |
| E | 10 | Gpt + CD20-TCB + | 30/5/1/1 | i.v. | 1 (once) + |
| | | CD19-4-1BBL (first) + | | | 14 (once weekly) + |
| | | CD19-CD28 (second) | | | 4 (once weekly) |
| | | | | | then 7 (once weekly) |
| F | 10 | Gpt + CD20-TCB + | 30/5/1/1 | i.v. | 1 (once) + |
| | | CD19-CD28 (first) + | | | 14 (once weekly) + |
| | | CD19-4-1BBL (second) | | | 4 (once weekly) |
| | | | | | then 7 (once weekly) |
| G | 10 | Gpt + CD20-TCB + | 30/5/1/1 | i.v. | 1 (once) + |
| | | CD19-CD28 + | | | 14 (once weekly) + |
| | | CD19-4-1BBL | | | 11 (once weekly) + |
| | | concomitant | | | 11 (once weekly) |

Tumor growth was measured three times weekly using a caliper and tumor volume was calculated as followed:

$${T}_{v} : ( {W}^{2} / 2 ) \times L ( W : Width , L : Length )$$

The study was terminated at day 120 after a total of 15 cycles of treatment.

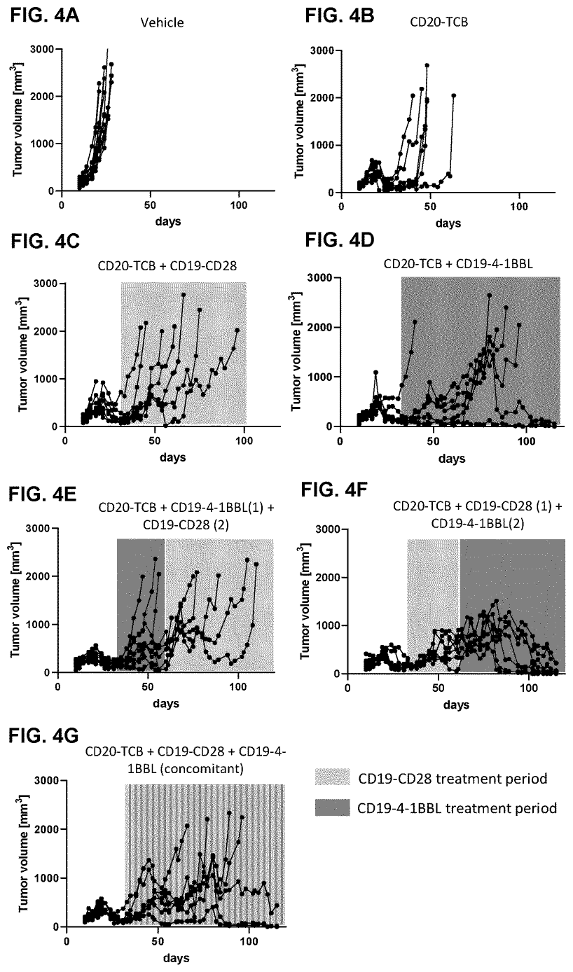

FIGS. 4A to 4G show the tumor growth as individual tumor growth kinetics per group and mouse. As described here, CD20-TCB monotherapy (FIG. 4B) initially induced strong tumor growth inhibition followed by tumor relapse in all animals treated. The combination treatment with CD19-CD28 (FIG. 4C) induced a mild delay of the tumor outgrowth in several mice. CD19-41BBL combination (FIG. 4D) revealed a more homogenous delay of the tumor relapse; however, most animals reached termination criteria (Tumor volume greater than 2000 mm³) before study termination. The treatment group receiving the triple combination injected concomitantly from day 38 onwards (FIG. 4G) did not show any further improvement in the increased duration of treatment activity as compared to the CD19-4-1BBL only combination arm. Interestingly, the group receiving an alternating treatment regime starting with CD19-CD28 for the first four cycles followed by CD19-4-1BBL combination treatment until study termination (FIG. 4F) resulted in complete tumor control over 120 days in all animals treated. In contrast, the alternation starting with CD19-4-1BBL followed by CD19-CD28 did not show this tumor control (FIG. 4E).

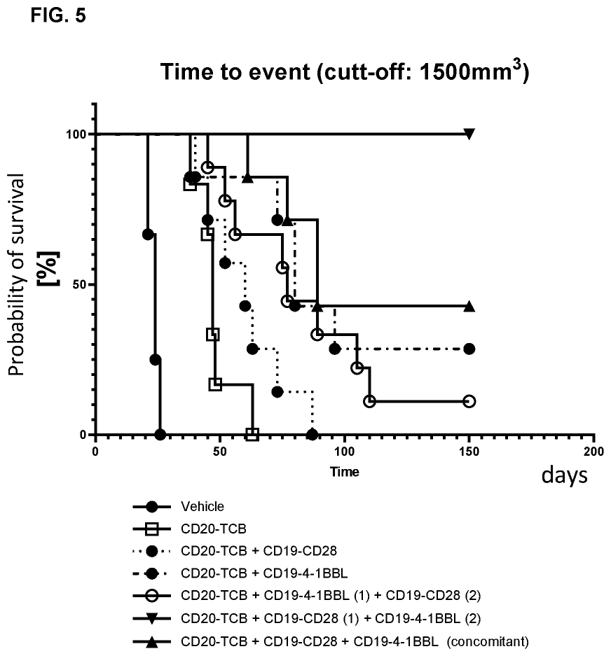

The time to event analysis (FIG. 5) using a tumor volume cut-off of 1500 m³ shows the strong synergistic effects for the combination of glofitamab with CD19-CD28 and CD19-4-1BBL when CD19-CD28 is given the first 4 treatment cycles and CD19-4-1BBL is given in the following treatment cycles.

### Example 6

Efficacy Study to Evaluate the Triple Combination Effect of CD20-TCB with CD19-4-1BBL and CD19-CD28 in Humanized BRGS-CD47 Mice

A second efficacy study was carried out in humanized BRGS-CD47 mice and aimed to evaluate the potential triple combination effect of CD20-TCB with CD19-CD28 and CD19-41BBL in a CD19/CD20-positive human lymphoma model when the combination treatment started a week earlier.

Human OCI-Ly18 (Diffuse large B cell lymphoma; DLBCL) were originally obtained from ATCC and after expansion deposited in the Roche Glycart internal cell bank. Cells were cultured in RPMI containing 10% FCS and 1× Glutamax. The cells were cultured at 37° C. in a water-saturated atmosphere at 5% CO₂. 50 microliters cell suspension (5×10⁶ NALM6 cells) mixed with 50 microliters Matrigel were injected subcutaneously in the flank of anaesthetized mice with a 22G to 30G needle.

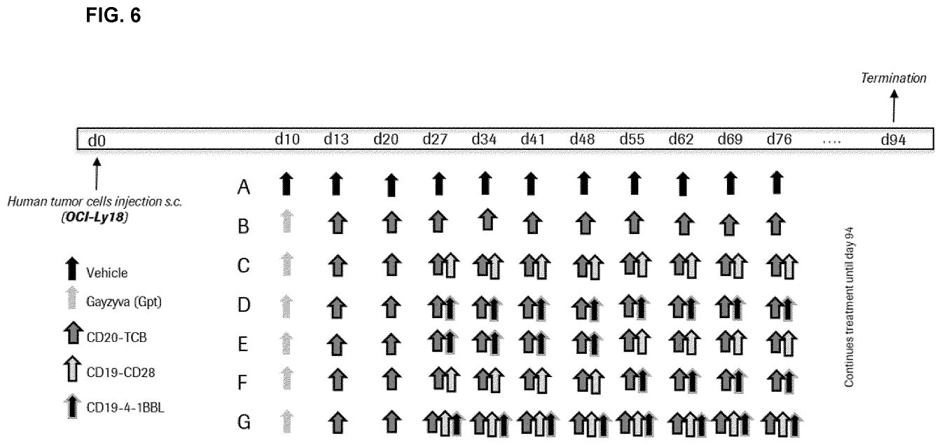

Female humanized BRGS-CD47 mice were generated by an injection of human hematopoietic stem cells at an age of 4-5 weeks at the Jackson Laboratory. Upon confirmation of engraftment, the humanized mice were shipped to Roche at an age of 15-16 weeks. Upon arrival, mice were maintained for one week to get accustomed to the new environment and for observation. Continuous health monitoring was carried out on a regular basis and mice were maintained under specific-pathogen-free condition with daily cycles of 12 h light/12 h darkness according to committed guidelines (GV-Solas; Felasa; TierschG). The experimental study protocol was reviewed and approved by local government (ZH181/2020). Mice were injected with tumor cells s.c. as described (FIG. 6) and treated with the compounds or Histidine buffer (Vehicle; Group A) when tumor size reached appr. 250 mm³ (at day 10). All mice were injected i.v. with 200 μl of the appropriate solution. To obtain the proper amount of compounds per 200 μl, the stock solutions (Table 1) were diluted with Histidine buffer when necessary. For combination treatments (Groups C to G), antibodies were mixed and injected concomitantly. All combination treatments started at day 27 upon tumor cell injection after receiving GAZYVA and two cycles of CD20-TCB. As displayed in FIG. 6, Group C received a combination with CD19-CD28 and Group D with CD19-4-1BBL. Group G received a triple combination injected concomitantly from day 27 onwards. Groups E and F received an alternating treatment schedule where treatment was either started with CD19-CD28 (Group F) or with CD19-4-1BBL (Group E) for four cycles followed by the injection of the other costimulatory molecule for the rest of all treatment cycles.

TABLE 3
Compositions used in this experiment

| | Formulation | Concentration |
|---|---|---|
| Compound | buffer | (mg/mL) |
| GAYZYVA | 20 mM Histidine, | 25.8 |
| (ID: P1AD3543-006) | 140 mM NaCl, | (=stock |
| | pH 6.0 | solution) |
| CD20-TCB | 20 mM Histidine, | 19.7 |
| (ID: P1AA4006-37290) | 140 mM NaCl, | (=stock |
| | pH 6.0 | solution) |
| CD19-CD28 | 20 mM Histidine, | 26.11 |
| (ID: P1AF0175-38963) | 140 mM NaCl, | (=stock |
| | pH 6.0 | solution) |
| CD19-4-1BBL | 20 mM Histidine, | 23.9 |
| (ID: P1AA0727-120602) | 140 mM NaCl, | (=stock |
| | pH 6.0 | solution) |

TABLE 4
Groups and their treatment regimen

| | No. of | | Dose | Injection | No. of |
|---|---|---|---|---|---|
| Group | animals | Compound | (mg/kg) | route | treatments |
| A | 10 | Vehicle | — | i.v. | 13 (once weekly) |
| B | 10 | Gpt + CD20-TCB | 30/5 | i.v. | 1 (once) + |
| | | | | | 12 (once weekly) |
| C | 10 | Gpt + CD20-TCB + | 30/5/1 | i.v. | 1 (once) + |
| | | CD19-CD28 | | | 12 (once weekly) + |
| | | | | | 11 (once weekly) |
| D | 10 | Gpt + CD20-TCB + | 30/5/1 | i.v. | 1 (once) + |
| | | CD19-4-1BBL | | | 12 (once weekly) + |
| | | | | | 10 (once weekly) |
| E | 10 | Gpt + CD20-TCB + | 30/5/1/1 | i.v. | 1 (once) + |
| | | CD19-4-1BBL (first) + | | | 12 (once weekly) + |
| | | CD19-CD28 (second) | | | 4 (once weekly) |
| | | | | | then 6 (once weekly) |
| F | 10 | Gpt + CD20-TCB + | 30/5/1/1 | i.v. | 1 (once) + |
| | | CD19-CD28 (first) + | | | 12 (once weekly) + |
| | | CD19-4-1BBL (second) | | | 4 (once weekly) |
| | | | | | then 6 (once weekly) |
| G | 10 | Gpt + CD20-TCB + | 30/5/1/1 | i.v. | 1 (once) + |
| | | CD19-CD28 + | | | 12 (once weekly) + |
| | | CD19-4-1BBL | | | 10 (once weekly) + |
| | | concomitant | | | 10 (once weekly) |

Tumor growth was measured three times weekly using a caliper and tumor volume was calculated as followed:

$${T}_{v} : ( {W}^{2} / 2 ) \times L ( W : Width , L : Length )$$

The study was terminated at day 94 after a total of 13 cycles of treatment.

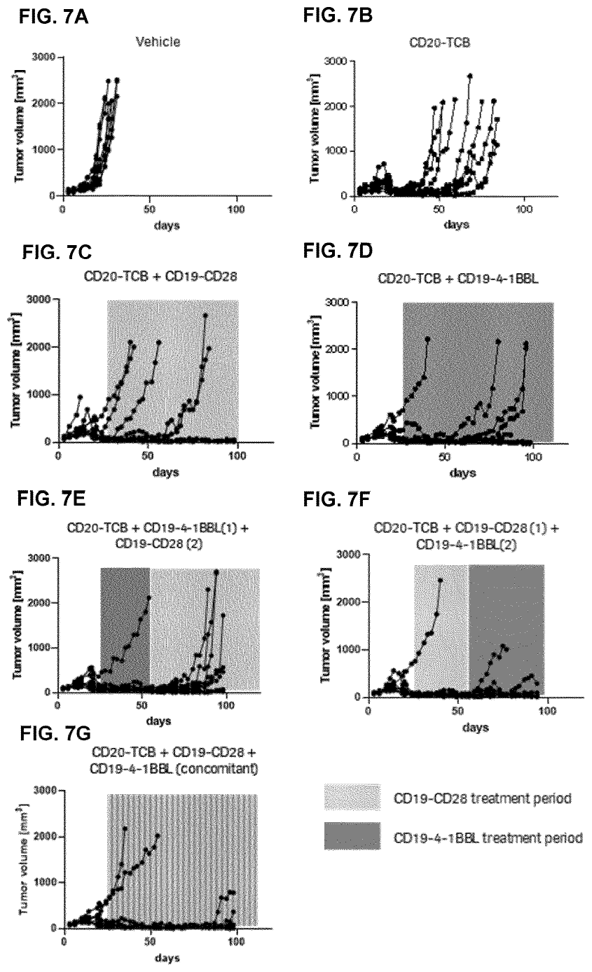

FIGS. 7A to 7G show the tumor growth as individual tumor growth kinetics per group and mouse. As described here, CD20-TCB monotherapy (FIG. 7B) initially induced strong tumor growth inhibition followed by tumor relapse in all animals treated. The combination treatment with CD19-CD28 (FIG. 7C) induced a delay of the tumor outgrowth in several mice and tumor control in a view mice. CD19-41BBL combination (FIG. 7D) revealed a similar delay of the tumor relapse. The treatment group receiving the triple combination injected concomitantly from day 27 onwards (FIG. 7G) did show a superior tumor control with increased duration of treatment activity as compared to the CD19-4-1BBL and CD19-CD28 only combination arm. The tumor control in this concomitant group is likely stronger in this experiment due to earlier start of combination treatment. The group receiving an alternating treatment regime starting with CD19-CD28 for the first four cycles followed by CD19-4-1BBL combination treatment until study termination (FIG. 7F) resulted in similar tumor control as the concomitant administration over 94 days. In contrast, the alternation starting with CD19-4-1BBL followed by CD19-CD28 did not show this pronounced tumor control (FIG. 7E).

1. An anti-CD20/anti-CD3 bispecific antibody in combination with an anti-CD19/anti-CD28 bispecific antibody and a CD19-targeted 4-1BB (CD137) agonist for use in a combination therapy for the treatment of B-cell proliferative disorders.

2. The use of an anti-CD20/anti-CD3 bispecific antibody in combination with an anti-CD19/anti-CD28 bispecific antibody and an anti-CD19-targeted 4-1BB (CD137) agonist in the manufacture of a medicament for use in a combination therapy for the treatment of B-cell proliferative disorders.

3. A method of treating B-cell cancer in an individual in need thereof comprising administering to said individual a combination therapy comprising an anti-CD20/anti-CD3 bispecific antibody in combination with an anti-CD19/anti-CD28 bispecific antibody and an anti-CD19-targeted 4-1BB (CD137) agonist.

4. A kit for use in a combination therapy comprising a first medicament comprising an anti-CD20/anti-CD3 bispecific antibody, a second medicament comprising an anti-CD19/anti-CD28 bispecific antibody and a third medicament comprising a CD19-targeted 4-1BB (CD137) agonist, and optionally further comprising a package insert comprising instruction for administration of the first medicament in combination with the second medicament for treating cancer in an individual.

5. A medicament comprising an anti-CD20/anti-CD3 bispecific antibody for treating B-cell proliferative disorders, wherein the anti-CD20/anti-CD3 bispecific antibody is used in combination with an anti-CD19/anti-CD28 bispecific antibody and a CD19-targeted 4-1BB (CD137) agonist.

6. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the combination therapy comprises a first treatment regimen with the anti-CD20/anti-CD3 bispecific antibody in combination with an anti-CD19/anti-CD28 bispecific antibody and a second treatment regimen with the anti-CD20/anti-CD3 bispecific antibody in combination with an CD19-targeted 4-1BB (CD137) agonist.

7. The anti-CD20/anti-CD3 bispecific antibody in combination with an anti-CD19/anti-CD28 bispecific antibody and a CD19-targeted 4-1BB (CD137) agonist for use, the use, the method, the kit or medicament according to claim 5, wherein the first treatment regimen comprises 1 to 5 treatment cycles and the second treatment regimen starts with the following treatment cycle.

8. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to claim 7 or 8, wherein the first treatment regimen comprises 4 treatment cycles and the second treatment regimen starts with the treatment cycle 5.

9. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to claim 6, wherein there is a time interval of one week between the end of the first treatment regimen and the start of the second treatment regimen.

10. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein a pretreatment with an Type II anti-CD20 antibody, preferably obinutuzumab, is performed prior to the combination therapy with an anti-CD19/anti-CD28 bispecific antibody and a CD19-targeted 4-1BB (CD137) agonist, and wherein the period of time between the pretreatment and said combination therapy is sufficient for the reduction of B-cells in the individual in response to the Type II anti-CD20 antibody, preferably obinutuzumab.

11. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the CD19-targeted 4-1BB agonist comprises three ectodomains of 4-1BBL each comprising an amino acid sequence selected from the group consisting of SEQ ID NO:1, SEQ ID NO: 2, SEQ ID NO: 3, SEQ ID NO:4, SEQ ID NO:5, SEQ ID NO: 6, SEQ ID NO:7, SEQ ID NO:8 and SEQ ID NO: 9, particularly the amino acid sequence of SEQ ID NO:5.

12. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the CD19-targeted 4-1BB agonist comprises a Fc domain, particularly an IgG1 or IgG4 Fc domain, that comprises one or more amino acid substitution that reduces or eliminates binding to an Fc receptor and/or effector function.

13. The anti-CD20/anti-CD3 bispecific antibody in combination with an anti-CD19/anti-CD28 bispecific antibody and a CD19-targeted 4-1BB (CD137) agonist for use, the use, the method, the kit or medicament according to any one of the preceding claims, wherein the CD19-targeted 4-1BB agonist comprises an antigen binding domain capable of specific binding to CD19 comprising a heavy chain variable region (V_{H}CD19) comprising (i) CDR-H1 comprising the amino acid sequence of SEQ ID NO:10, (ii) CDR-H2 comprising the amino acid sequence of SEQ ID NO:11, and (iii) CDR-H3 comprising the amino acid sequence of SEQ ID NO:12, and a light chain variable region (V_{L}CD19) comprising (iv) CDR-L1 comprising the amino acid sequence of SEQ ID NO:13, (v) CDR-L2 comprising the amino acid sequence of SEQ ID NO:14, and (vi) CDR-L3 comprising the amino acid sequence of SEQ ID NO: 15.

14. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the CD19-targeted 4-1BB agonist comprises an antigen binding domain capable of specific binding to CD19 comprising a heavy chain variable region (V_{H}CD19) comprising an amino acid sequence of SEQ ID NO:16 and a light chain variable region (V_{L}CD19) comprising an amino acid sequence of SEQ ID NO:17.

15. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the CD19-targeted 4-1BB agonist comprises
(a) a first polypeptide, comprising (a1) the first ectodomain of 4-1BBL or fragment thereof, fused at its C-terminus to the N-terminus of the second ectodomain of 4-1BBL or fragment thereof, (a2) the second ectodomain of 4-1BBL or fragment thereof, fused at its C-terminus to the N-terminus of the CL domain, (a3) the CL domain, fused at its C-terminus to the N-terminus of one of the subunits (e.g. the first subunit) of the Fc domain, and (a4) one of the subunits (e.g. the first subunit) of the Fc domain;
(b) a second polypeptide, comprising (b1) the third ectodomain of 4-1BBL or fragment thereof, fused at its C-terminus to the N-terminus of the CH1 domain, and (b2) the CH1 domain;
(c) a third polypeptide, comprising (c1) the heavy chain of the Fab molecule that binds to CD19, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising the light chain of the Fab molecule that binds to CD19.

16. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the CD19-targeted 4-1BB agonist comprises a first polypeptide comprising an amino acid sequence that is at least about 95%, 96%, 97%, 98%, 99% or 100% identical to the amino acid sequence of SEQ ID NO: 18, a second polypeptide comprising an amino acid sequence that is at least about 95%, 96%, 97%, 98%, 99% or 100% identical to the amino acid sequence of SEQ ID NO: 19, a third polypeptide comprising an amino acid sequence that is at least about 95%, 96%, 97%, 98%, 99% or 100% identical to the amino acid sequence of SEQ ID NO: 20, and a fourth polypeptide comprising an amino acid sequence that is at least about 95%, 96%, 97%, 98%, 99% or 100% identical to the amino acid sequence of SEQ ID NO: 21.

17. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the CD19-targeted 4-1BB agonist is englumafusp alfa.

18. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD20/anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}CD20) and a light chain variable region (V_{L}CD20).

19. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the first antigen binding domain comprises a heavy chain variable region (V_{H}CD3) comprising CDR-H1 sequence of SEQ ID NO:22, CDR-H2 sequence of SEQ ID NO:23, and CDR-H3 sequence of SEQ ID NO:24; and/or a light chain variable region (V_{L}CD3) comprising CDR-L1 sequence of SEQ ID NO:25, CDR-L2 sequence of SEQ ID NO:26, and CDR-L3 sequence of SEQ ID NO:27.

20. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the first antigen binding domain comprises a heavy chain variable region (V_{H}CD3) comprising the amino acid sequence of SEQ ID NO:28 and/or a light chain variable region (V_{L}CD3) comprising the amino acid sequence of SEQ ID NO:29.

21. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the second antigen binding domain comprises a heavy chain variable region (V_{H}CD20) comprising CDR-H1 sequence of SEQ ID NO:30, CDR-H2 sequence of SEQ ID NO:31, and CDR-H3 sequence of SEQ ID NO:32, and/or a light chain variable region (V_{L}CD20) comprising CDR-L1 sequence of SEQ ID NO:33, CDR-L2 sequence of SEQ ID NO:34, and CDR-L3 sequence of SEQ ID NO:35.

22. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the second antigen binding domain comprises a heavy chain variable region (V_{H}CD20) comprising the amino acid sequence of SEQ ID NO:36 and/or a light chain variable region (V_{L}CD20) comprising the amino acid sequence of SEQ ID NO:37.

23. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD20/anti-CD3 bispecific antibody comprises a third antigen binding domain that binds to CD20.

24. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD20/anti-CD3 bispecific antibody comprises a first polypeptide comprising an amino acid sequence that is at least about 95%, 96%, 97%, 98%, 99% or 100% identical to the amino acid sequence of SEQ ID NO: 38, a second polypeptide comprising an amino acid sequence that is at least about 95%, 96%, 97%, 98%, 99% or 100% identical to the amino acid sequence of SEQ ID NO: 39, a third polypeptide comprising an amino acid sequence that is at least about 95%, 96%, 97%, 98%, 99% or 100% identical to the amino acid sequence of SEQ ID NO: 40, and a fourth and fifth polypeptide both comprising an amino acid sequence that is at least about 95%, 96%, 97%, 98%, 99% or 100% identical to the amino acid sequence of SEQ ID NO: 41.

25. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD20/anti-CD3 bispecific antibody is glofitamab.

26. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a first antigen binding domain comprising a heavy chain variable region (V_{H}CD28) and a light chain variable region (V_{L}CD28), and a second antigen binding domain comprising a heavy chain variable region (V_{H}CD19) and a light chain variable region (V_{L}CD19).

27. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a first antigen binding domain comprising a heavy chain variable region (V_{H}CD28) comprising CDR-H1 sequence of SEQ ID NO:42, CDR-H2 sequence of SEQ ID NO:43, and CDR-H3 sequence of SEQ ID NO:44, and/or a light chain variable region (V_{L}CD28) comprising CDR-L1 sequence of SEQ ID NO:45, CDR-L2 sequence of SEQ ID NO:46, and CDR-L3 sequence of SEQ ID NO:47.

28. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a first antigen binding domain comprising a heavy chain variable region (V_{H}CD28) comprising the amino acid sequence of SEQ ID NO:48 and/or a light chain variable region (V_{L}CD28) comprising the amino acid sequence of SEQ ID NO: 49.

29. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a second antigen binding domain comprising a heavy chain variable region (V_{H}CD19) comprising CDR-H1 sequence of SEQ ID NO: 10, CDR-H2 sequence of SEQ ID NO:11, and CDR-H3 sequence of SEQ ID NO:12; and/or a light chain variable region (V_{L}CD19) comprising CDR-L1 sequence of SEQ ID NO:13, CDR-L2 sequence of SEQ ID NO: 14, and CDR-L3 sequence of SEQ ID NO:15.

30. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a second antigen binding domain comprising a heavy chain variable region (V_{H}CD19) comprising the amino acid sequence of SEQ ID NO:16 and/or a light chain variable region (V_{L}CD19) comprising the amino acid sequence of SEQ ID NO:17.

31. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a Fc domain, particularly an IgG1 or IgG4 Fc domain, that comprises one or more amino acid substitution that reduces or eliminates binding to an Fc receptor and/or effector function.

32. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the anti-CD19/anti-CD28 bispecific antibody comprises a first polypeptide comprising an amino acid sequence that is at least about 95%, 96%, 97%, 98%, 99% or 100% identical to the amino acid sequence of SEQ ID NO: 50, a second polypeptide comprising an amino acid sequence that is at least about 95%, 96%, 97%, 98%, 99% or 100% identical to the amino acid sequence of SEQ ID NO: 51, a third polypeptide comprising an amino acid sequence that is at least about 95%, 96%, 97%, 98%, 99% or 100% identical to the amino acid sequence of SEQ ID NO: 52, and a fourth polypeptide comprising an amino acid sequence that is at least about 95%, 96%, 97%, 98%, 99% or 100% identical to the amino acid sequence of SEQ ID NO: 53.

33. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the combination therapy is administered at intervals from about one week to three weeks.

34. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the B-cell proliferative disorder is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), Multiple myeloma (MM) and Hodgkin lymphoma (HL).

35. The anti-CD20/anti-CD3 bispecific antibody in combination with an anti-CD19/anti-CD28 bispecific antibody and a CD19-targeted 4-1BB (CD137) agonist for use, the use, the method, the kit or the medicament according to any one of the preceding claims, wherein the B-cell proliferative disorder is Non-Hodgkin lymphoma (NHL) or diffuse large B-cell lymphoma (DLBCL).


SEQUENCE LISTING (68 sequences)

SEQ ID NO: 1 - protein, 184 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 170 aa, synthetic construct
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 175 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 203 aa, synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 178 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 6 - protein, 164 aa, synthetic construct
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 7 - protein, 169 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 8 - protein, 197 aa, synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 9 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 5 aa, synthetic construct
DYIMH

SEQ ID NO: 11 - protein, 17 aa, synthetic construct
YINPYNDGSKYTEKFQG

SEQ ID NO: 12 - protein, 12 aa, synthetic construct
GTYYYGPQLFDY

SEQ ID NO: 13 - protein, 16 aa, synthetic construct
KSSQSLETSTGTTYLN

SEQ ID NO: 14 - protein, 7 aa, synthetic construct
RVSKRFS

SEQ ID NO: 15 - protein, 9 aa, synthetic construct
LQLLEDPYT

SEQ ID NO: 16 - protein, 121 aa, synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSS

SEQ ID NO: 17 - protein, 112 aa, synthetic construct
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 18 - protein, 708 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 19 - protein, 291 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 20 - protein, 449 aa, synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 21 - protein, 219 aa, synthetic construct
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 5 aa, synthetic construct
TYAMN

SEQ ID NO: 23 - protein, 19 aa, synthetic construct
RIRSKYNNYATYYADSVKG

SEQ ID NO: 24 - protein, 14 aa, synthetic construct
HGNFGNSYVSWFAY

SEQ ID NO: 25 - protein, 14 aa, synthetic construct
GSSTGAVTTSNYAN

SEQ ID NO: 26 - protein, 7 aa, synthetic construct
GTNKRAP

SEQ ID NO: 27 - protein, 9 aa, synthetic construct
ALWYSNLWV

SEQ ID NO: 28 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 29 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 30 - protein, 5 aa, synthetic construct
YSWIN

SEQ ID NO: 31 - protein, 17 aa, synthetic construct
RIFPGDGDTDYNGKFKG

SEQ ID NO: 32 - protein, 10 aa, synthetic construct
NVFDGYWLVY

SEQ ID NO: 33 - protein, 16 aa, synthetic construct
RSSKSLLHSNGITYLY

SEQ ID NO: 34 - protein, 7 aa, synthetic construct
QMSNLVS

SEQ ID NO: 35 - protein, 9 aa, synthetic construct
AQNLELPYT

SEQ ID NO: 36 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 37 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 38 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 39 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 40 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 5 aa, synthetic construct
SYYIH

SEQ ID NO: 43 - protein, 17 aa, synthetic construct
SIYPGNVQTNYNEKFKD

SEQ ID NO: 44 - protein, 11 aa, synthetic construct
SHYGLDFNFDV

SEQ ID NO: 45 - protein, 11 aa, synthetic construct
HASQNIYVYLN

SEQ ID NO: 46 - protein, 7 aa, synthetic construct
KASNLHT

SEQ ID NO: 47 - protein, 9 aa, synthetic construct
QQGQTYPYT

SEQ ID NO: 48 - protein, 120 aa, synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSS

SEQ ID NO: 49 - protein, 107 aa, synthetic construct
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIK

SEQ ID NO: 50 - protein, 448 aa, synthetic construct
QVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 51 - protein, 214 aa, synthetic construct
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 442 aa, synthetic construct
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 53 - protein, 228 aa, synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 557 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 55 - protein, 296 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSS

SEQ ID NO: 56 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 59 - protein, 198 aa, synthetic construct
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 60 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 61 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 62 - protein, 5 aa, Homo sapiens
EPKSC

SEQ ID NO: 63 - protein, 107 aa, Homo sapiens
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 64 - protein, 106 aa, Homo sapiens [sequence omitted]

SEQ ID NO: 65 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 66 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 67 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 68 - protein, 10 aa, synthetic construct
GGGGSGGGGS